(19) United States
(12) Patent Application Publication
(10) Pub. No.: US 2009/0222282 A1
(43) Pub. Date: Sep. 3, 2009

(54) Method for Personalized Diet Design

(75) Inventors: Jose M. Ordovas, Framingham, MA (US); Dolores Corella, Valencia (ES)

Correspondence Address: DAVID S. RESNICK, NIXON PEABODY LLP, 100 SUMMER STREET, BOSTON, MA, 02110-2131, US

(73) Assignee: TRUSTEES OF TUFTS COLLEGE, Medford, MA (US)

(21) Appl. No.: 12/066,902

(22) PCT Filed: Sep. 15, 2006
(86) PCT No.: PCT/US06/35941

§ 371 Date: Oct. 2, 2008

Related U.S. Application Data
(60) Provisional application No. 60/717,345, filed on Sep. 15, 2005.

(51) Int. Cl. G06Q 50/00; C12Q 1/68

(52) U.S. Cl. 705/2; 435/6

(57) ABSTRACT

The present invention is directed to methods of developing dietary guidelines, particularly associated with dietary consumption of fatty acids. We discovered an association of a polymorphic marker with regulation of plasma lipid levels in response to dietary intake of polyunsaturated fatty acids. Accordingly, the present invention provides methods for providing individualized guidance in design of dietary interventions to alter plasma lipid profiles by genotyping APOA5 locus, wherein the presence of APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles, is indicative of the individual being susceptible to altered plasma lipid levels in response to intake of n-6 polyunsaturated fatty acids.

### CROSS-REFERENCE TO RELATED APPLICATIONS

This application claims benefit under 35 U.S.C. 119(e) of the U.S. provisional application Ser. No. 60/717,345, filed on Sep. 15, 2005, the content of which is herewith incorporated by reference in its entirety.

### GOVERNMENT SUPPORT

The present invention was supported by the National Heart, Lung and Blood Institute Contract N01-HC-25195, and National Institutes of Health/NHLBI grant no. HL54776, contracts 53-K06-5-10 and 58-1950-9-001 from the U.S. Department of Agriculture Research Service. The Government of the United States has certain rights thereto.

### BACKGROUND

The present invention is related to genetic tests and methods. Particularly, the invention is directed to methods to assess an individual's likelihood of responsiveness to dietary lipid profile management by genetically classifying individuals as likely susceptible or likely resistant to altered plasma lipid profile, for example, plasma triglyceride (TG) concentration, plasma remnant-like particle (RLP) concentration, plasma very low density lipoprotein (VLDL) and plasma low density lipoprotein (LDL) size, in response to polyunsaturated fatty acids.

Dietary intervention to modulate plasma lipid profile is an important aspect of disease prevention programs, such as cardiovascular disease prevention programs. Reduction of total cholesterol, particularly low density lipoprotein (LDL), levels as well as plasma triglyceride levels is known to be associated with reduced disease risk.

It is well recognized that the genetic background of individuals affects their responses to pharmaceutical as well as dietary interventions. It would be useful to identify genetic polymorphisms that are associated with predictable responses to dietary fat intake. Such polymorphisms could provide more individualized guidance in design of dietary interventions to alter plasma lipid profiles.

### SUMMARY

The present invention provides an association of a polymorphic marker with regulation of plasma lipid levels in response to dietary intake of polyunsaturated fatty acids. Accordingly, the present invention provides methods for providing individualized guidance in design of dietary interventions to alter plasma lipid profiles by genotyping APOA5 locus, wherein the presence of APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles, is indicative of the individual being susceptible to altered plasma lipid levels in response to intake of n-6 polyunsaturated fatty acids.

We have now discovered that in carriers of the APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles, higher n-6 polyunsaturated fatty acid (PUFA) intake, but not n-3 PUFA intake, significantly alters plasma lipid profile, for example, it increases plasma triglyceride (TG) and plasma remnant-like particle (RLP) concentrations and VLDL size, and decreases LDL size. These results demonstrate that a diet high in n-6 PUFA is related to a more atherogenic lipid profile in carriers of the APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles.

Accordingly, we provide a method for personalized diet design based on screening for the APOA5 polymorphic alleles in a biological sample from the individual, wherein the presence of any one or more of APOA5 alleles including, but not limited to APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles is indicative of the individual being susceptible of increased altered plasma lipid profile, for example, increased plasma triglyceride (TG) concentration, plasma remnant-like particle (RLP) concentration, and increased plasma very low density lipoprotein (VLDL) size and decreased plasma low density lipoprotein (LDL) size, upon intake of n-6 polyunsaturated fatty acids, and wherein the absence of the APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles is indicative of the individual being less susceptible of increased plasma triglyceride (TG) concentration, plasma remnant-like particle (RLP) concentration, plasma very low density lipoprotein (VLDL) and plasma low density lipoprotein (LDL) size upon intake of n-6 polyunsaturated fatty acids. Thus, to improve lipid profile in an individual carrying APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles, the individual should avoid consumption of n-6 PUFAs, and preferably substitute n-6 PUFAs with n-3 PUFAs, while individuals who do not carry the APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles, can consume both n-3 and n-6 PUFAs without it adversely affecting their plasma lipid profile, such as increased plasma triglyceride (TG) concentration, increased plasma remnant-like particle (RLP) concentration, and increased plasma very low density lipoprotein (VLDL) size and decreased plasma low density lipoprotein (LDL) size.

The method of the present invention provides a simple assay that can help a nutritionist or a clinician to create effective and individualized diets for altering, preferably reducing, the plasma triglyceride levels, and therefore reducing the risk factors for high lipid concentration associated diseases such as atherosclerosis and other cardiovascular diseases.

In one embodiment, the invention provides a kit for determining individual's response to n-6 PUFAs. The kit comprises genotyping means or a genotyping system for APOA5 locus for one or more polymorphisms, particularly for detecting APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles in a biological sample, and instructions, which explain that an individual carrying APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles is at increased risk of altered plasma lipid profile, particularly increased plasma remnant-like particle (RLP) concentration, and increased plasma very low density lipoprotein (VLDL) size and decreased plasma low density lipoprotein (LDL) size, if consuming n-6 PUFAs, and/or that an individual not carrying the APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles can likely consume n-6 PUFAs without it adversely affecting their plasma lipid profile.

The genotyping system or means may include nucleic acid probes attached to chips or beads. The genotyping system or means may also be a part of a chip or system that incorporates other lipid profile predicting polymorphisms on the same chip or bead mixture.

In an alternative embodiment, one looks at the presence of homozygotes in the APOA5 locus, including but not limited to APOA5−1131T/T, APOA5−3A/A, APOA5 IVS3+476G/G, and APOA5 1259T/T alleles or alleles that are found to be in tight linkage disequilibrium with these alleles, wherein presence of any one of these homozygotes is indicative of the individual being less susceptible to developing an adverse plasma lipid profile, such as increased plasma remnant-like particle (RLP) concentration, and increased plasma very low density lipoprotein (VLDL) size and decreased plasma low density lipoprotein (LDL) size, in response to consumption of n-6 PUFAs. We have also discovered that the presence of the rare allele of the APOA5 56C>G (i.e. the G allele) is associated with a higher common carotid artery (CCA) catorid intimal thickness (IMT) when compared with the wild-type haplotype.

In addition, the rare allele of each APOA5 polymorphic locus −1131T>C, −3A>G, IVS3+476G>A, and 1259T>C variants and the haplotype defined by the presence of the rare alleles in these loci were each significantly associated with CCA IMT in obese participants.

Accordingly, the invention also provides a method of identifying individuals with higher risk for developing CCA IMT by analyzing at least one locus selected from −1131T>C, −3A>G, IVS3+476G>A, and 1259T>C for the rare allele, wherein the presence of the rare allele is indicative of the individual being at higher risk, particularly if the individual is obese, of developing CCA IMT. In one embodiment, one analyzes all four of these loci. In one embodiment, one uses loci in tight linkage disequilibrium with the alleles in said loci.

Accordingly, in one embodiment, the method for personalized diet design further comprises a step of determining whether one or more of the rare alleles in the loci −1131T>C, −3A>G, IVS3+476G>A, and 1259T>C are present and if so, advising the individual carrying such allele or a haplotype comprising such allele to particularly monitor their weight and keep it under the overweight or at least obese levels in order to decrease their risk for CCA IMT and associated diseases, such as atherosclerosis.

### BRIEF DESCRIPTION OF THE DRAWINGS

FIG. 1 shows mean remnant-like particle TG concentration (RLP-TG) in men and women combined by APOA5−1131T>C genotypes (open bar=TT, solid bar=TC+CC) and PUFA fat intake categories (PUFA<6% or PUFA>6%). Means were adjusted for sex, age, familial relationships, BMI, smoking, alcohol, diabetes status, estrogens, β-blockers, and energy. Error bars: standard error of means.

FIG. 2 shows predicted values of remnant-like particle TG concentration (RLP-TG) by APOA5−1131T>C genotypes (in men and women combined; n=1527) depending on the PUFA fat consumed (as a continuous variable). Predicted values were calculated from the regression models containing PUFA fat intake, −1131T>C polymorphism, their interaction term, and the potential confounders (sex, age, familial relationships, BMI, smoking, alcohol, diabetes status, estrogens, β-blockers, and energy). □=TT, *=C allele carriers.

FIGS. 3A-3B show mean remnant-like particle triglyceride concentration (RLP-TG) in men and women combined by PUFA categories (below and above the population mean) and APOA5−1131T>C genotypes (TT homozygote or −1131C carrier). Means were adjusted for sex, age, familial relationships, BMI, smoking, alcohol, diabetes status, estrogens, β-blockers, and energy. Error bars: standard error of means.

FIGS. 4A-4B show mean remnant-like particle cholesterol concentration (RLP-C) in men and women combined by PUFA categories of tertiles (grey=1st tertile, dark=2nd tertile, and solid=3th tertile) and APOA5−1131T>C genotypes (TT homozygote or −1131C carrier). Means were adjusted for sex, age, familial relationships, BMI, smoking, alcohol, diabetes status, estrogens, β-blockers, and energy. Error bars: standard error of means.

FIG. 5 shows predicted values from the regression of the adjusted common carotid artery (CCA) intimal medial thickness on BMI within each APOA5 haplotype-genotype group, and p value for the interaction between APOA5 haplotype-genotype and BMI. Adjusted for age, sex, smoking, diabetes, systolic blood pressure, hypertension treatment, triglycerides, HDL and LDL cholesterol.

FIG. 6 shows multivariable adjusted common carotid artery intimal medial thickness (CCA IMT) according to the APOA5 haplotype-genotype groups and obesity, and p value for the interaction between these haplotypes and obesity. Adjusted for age, sex, smoking, diabetes, systolic blood pressure, hypertension treatment, triglycerides, HDL and LDL cholesterol.

FIGS. 7A-7D show predicted values from the regression of the adjusted common carotid artery (CCA) intimal medial thickness on BMI according to the individual APOA5 genotypes, and p value for the interaction between these genotypes and BMI. Adjusted for age, sex, smoking, diabetes, systolic blood pressure, hypertension treatment, triglycerides, HDL and LDL cholesterol.

FIGS. 8A-8D show multivariable adjusted common carotid artery intimal medial thickness (CCA IMT) according to the analyzed individual APOA5 genotypes and obesity, and p value for the interaction between these genotypes and obesity. Adjusted for age, sex, smoking, diabetes, systolic blood pressure, hypertension treatment, triglycerides, HDL and LDL cholesterol.

### DETAILED DESCRIPTION

We have discovered APOA5 polymorphisms that are associated with dietary responses to fat intake. We have discovered that APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles are associated with altered plasma lipid profile, particularly increased plasma remnant-like particle (RLP) concentration, and increased plasma very low density lipoprotein (VLDL) size and decreased plasma low density lipoprotein (LDL) size in response to consumption of n-6 polyunsaturated fatty acids (PUFA), thereby being susceptible to more atherogenic lipid profile when consuming n-6 PUFAs. The carriers of APOA5−1131T, APOA5−3A, APOA5 IVS3+476G, and APOA5 1259T alleles or alleles that are found to be in tight linkage disequilibrium with these alleles, did not respond to n-6 PUFAs similar plasma lipid profile alteration. These individuals are thus not susceptible of developing atherogenic plasma lipid profile in response to consumption of n-6 PUFAs.

Apolipoprotein A5 (APOA5) has been identified as an important player in plasma triglycelide lipid (TLR) metabolism (9). However, its precise mechanisms of action as well as its dietary modulation by fat remains to be defined. It is known that APOA5 expression is upregulated in liver regeneration after rat hepatectomy (10) suggesting that is plays a role in very-low density lipoprotein (VLDL) assembly (11,12). APOA5 is found preferentially on high-density lipoprotein (HDL) but is thought to transfer to VLDL during the postprandial state (13). Furthermore, APOA5 has been shown to activate lipoprotein lipase (LPL), a regulator of TRL metabolism (12,14).

Several common APOA5 single nucleotide polymorphisms (SNPs) have been identified and their variant alleles associated with increased plasma TG in several populations (15-19). In addition, we have earlier reported associations with increased RLP-TG, RLP-C and VLDL concentrations in the Framingham Heart Study (19).

Among dietary factors, polyunsaturated fatty acids (PUFA) are one of the most important modulators of gene expression (20). In addition, although PUFA intake has been recommended as a replacement of saturated fat to decrease cardio vascular disease (CVD) risk, there are some conflicting observations (21,22). Moreover, there is discussion about the specific contributions of the n-3 and n-6 families to the potential CVD protection, with several studies suggesting that their cardiovascular benefit is provided primarily by dietary n-3 PUFA (23-25).

We have now discovered that in carriers of the APOA5−1131C, APOA5−3G, APOA5 IVS3+476A, and APOA5 1259C allele or alleles that are found to be in tight linkage disequilibrium with APOA5−1131C, APOA5−3G, APOA5 IVS3+476A, and APOA5 1259C alleles, are at increased risk of developing altered and more atherogenic plasma lipid profile, when consuming n-6 polyunsaturated fatty acids (PUFA), but not when consuming n-3 PUPA. The more atherogenic lipid profile means at least increases plasma triglyceride (TG) and plasma remnant-like particle (RLP) concentrations and VLDL size, and decreased LDL size. Such more atherogenic lipid profile increases the individual's risk of developing diseases, such as atherosclerosis and other cardiovascular diseases known to be associated with atherogenic lipid profile.

Accordingly, the present invention provides a method for individualized dietary advice comprising determining the alleles at the APOA5 locus from a biological sample of an individual, wherein the presence of one or two APOA5−1131C, APOA5−3G, APOA5 IVS3+476A, and APOA5 1259C alleles, or alleles that are found to be in tight linkage disequilibrium with APOA5−1131C, APOA5−3G, APOA5 IVS3+476A, and APOA5 1259C alleles, indicates that the individual should limit or eliminate consumption of n-6 PUFAs. This is because consumption of n-6 PUFAs in an individual carrying one or two APOA5−1131C, APOA5−3G, APOA5 IVS3+476A, and APOA5 1259C alleles adversely affects his/her plasma lipid profile.

In one embodiment, the individuals with one or two APOA5−1131C, APOA5−3G, APOA5 IVS3+476A, and APOA5 1259C alleles, or alleles that are found to be in tight linkage disequilibrium with APOA5−1131C, APOA5−3G, APOA5 IVS3+476A, and APOA5 1259C alleles

As used herein, a “tight linkage disequilibrium” means a polymorphic marker that co-segregates 100% with the allele “C” in the APOA5−1131 locus, allele “G” in APOA5−3, allele “A” in APOA5 IVS3+476 locus, and allele “C” in APOA5 1259 locus. Therefore, any tightly linked polymorphic marker discovered by, for example, in-silico searches or by re-sequencing of carriers of the APOA5−1131C, APOA5−3G, APOA5 IVS3+476A, and APOA5 1259C alleles, could be also used.

The polymorphisms are analyzed from nucleic acids isolated from any biological sample taken from an individual. Biological sample used as a source material for isolating the nucleic acids in the instant invention include, but are not limited to solid materials (e.g., tissue, cell pellets, biopsies, hair follicle samples, buccal smear or swab) and biological fluids (e.g. blood, saliva, amniotic fluid, mouth wash, urine). Any biological sample from a human individual comprising even one cell comprising nucleic acid, can be used in the methods of the present invention.

Nucleic acid molecules of the instant invention include DNA and RNA, for example genomic DNA, and can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001.

The APOA5 polymorphisms according to the present invention can be detected from the isolated nucleic acids using techniques including direct analysis of isolated nucleic acids such as Southern Blot Hybridization (DNA) or direct nucleic acid sequencing (Molecular Cloning: A Laboratory Manual, 3rd Ed., Sambrook and Russel, Cold Spring Harbor Laboratory Press, 2001).

An alternative method useful according to the present invention for direct analysis of the APOA5 polymorphisms is the INVADER® assay (Third Wave Technologies, Inc (Madison, Wis.). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535).

In one embodiment, a nucleic acid amplification, such as PCR based techniques are used. After nucleic acid amplification, the polymorphic nucleic acids can be identified using, for example direct sequencing with radioactively or fluorescently labeled primers; single-stand conformation polymorphism analysis (SSCP), denaturating gradient gel electrophoresis (DGGE); and chemical cleavage analysis, all of which are explained in detail, for example, in the Molecular Cloning: A Laboratory Manual, 3rd Ed., Sambrook and Russel, Cold Spring Harbor Laboratory Press, 2001.

The APOA5 polymorphisms are in one embodiment analyzed using methods amenable for automation such as the different methods for primer extension analysis. Primer extension analysis can be preformed using any method known to one skilled in the art including PYROSEQUENCING™ (Uppsala, Sweden); Mass Spectrometry including MALDI-TOF, or Matrix Assisted Laser Desorption Ionization-Time of Flight; genomic nucleic acid arrays (Shalon et al., Genome Research 6(7):639-45, 1996; Bernard et al., Nucleic Acids Research 24(8): 1435-42, 1996); solid-phase mini-sequencing technique (U.S. Pat. No. 6,013,431, Suomalainen et al. Mol. Biotechnol. June; 15(2):123-31, 2000); ion-pair high-performance liquid chromatography (Doris et al. J. Chromatogr. A May 8; 806(1):47-60, 1998); and 5′ nuclease assay or real-time RT-PCR (Holland et al. Proc Natl Acad Sci USA 88: 7276-7280, 1991), or primer extension methods described in the U.S. Pat. No. 6,355,433. Nucleic acids sequencing, for example using any automated sequencing system and either labeled primers or labeled terminator dideoxynucleotides can also be used to detect the polymorphisms. Systems for automated sequence analysis include, for example, Hitachi FMBIO® and Hitachi FMBIO® II Fluorescent Scanners (Hitachi Genetic Systems, Alameda, Calif.); Spectrumedix® SCE 9610 Fully Automated 96-Capillary Electrophoresis Genetic Analysis System (SpectruMedix LLC, State College, Pa.); ABI PRISM® 377 DNA Sequencer; ABI® 373 DNA Sequencer; ABI PRISM® 310 Genetic Analyzer; ABI PRISM® 3100 Genetic Analyzer; ABI PRISM® 3700 DNA Analyzer (Applied Biosystems, Headquarters, Foster City, Calif.); Molecular Dynamics FluorImager™ 575 and SI Fluorescent Scanners and Molecular Dynamics FluorImager™ 595 Fluorescent Scanners (Amersham Biosciences UK Limited, Little Chalfont, Buckinghamshire, England); GenomyxSC™ DNA Sequencing System (Genomyx Corporation (Foster City, Calif.); Pharmacia ALF™ DNA Sequencer and Pharmacia ALFexpress™ (Amersham Biosciences UK Limited, Little Chalfont, Buckinghamshire, England).

Nucleic acid amplification, nucleic acid sequencing and primer extension reactions for one nucleic acid sample can be performed in the same or separate reactions using the primers designed to amplify and detect the polymorphic APOA5 nucleotides.

In one embodiment, the invention provides a kit comprising one or more primer pairs capable of amplifying the APOA5 nucleic acid regions comprising the APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C polymorphic nucleotides of the present invention; buffer and nucleotide mix for the PCR reaction; appropriate enzymes for PCR reaction in same or separate containers as well as an instruction manual defining the PCR conditions, for example, as described in the Example below. The kit may further comprise nucleic acid probes to detect the APOA5 APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles. Primers may also be provided in the kit in either dry form in a tube or a vial, or alternatively dissolved into an appropriate aqueous buffer. The kit may also comprise primers for the primer extension method for detection of the specific APOA5−1131C, APOA5−3G, APOA5 IVS3+476A, and APOA5 1259C alleles or alleles that are found to be in tight linkage disequilibrium with APOA5−113° C., APOA5−3G, APOA5 IVS3+476A, and APOA5 1259C allelic polymorphisms as described above.

In one embodiment, the components of the kit are part of a kit providing for multiple plasma lipid metabolism regulation or cardiovascular disease risk associated genes and polymorphisms and or mutations known to one skilled in the art, in addition to detecting APOA5−1131C, APOA5−3G, APOA5 IVS3+476A, and APOA5 1259C allelic polymorphisms. Such other mutations and/or polymorphisms include, but are not limited to mutations and polymorphisms associated with weight regulation.

In one embodiment, the invention provides a kit for determining susceptibility to increased plasma lipid levels, such as increased plasma TG levels, using dietary intervention, including a n-6 PUFA containing diet. Such kit includes, for example, instructions that if an allele “C” at APOA5−1131 locus is detected in the tested individual, the individual is likely to be susceptible to increased plasma lipid, such as plasma TG levels if their diet would contain n-6 PUFAs, and if the individual does not carry allele “C”, n-6 PUFAs in the diet of that individual will likely not increase plasma lipid levels, such as TG levels in the individual. The kit also includes means to detect polymorphisms in the APOA5 locus, in one embodiment, the polymorphisms are APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles. The kit may also include only a detection means for detecting the APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles, wherein a negative result, for example, no PCR product, or no signal, is indicative of the individual's plasma lipid profile, being likely not adversely affected by the presence of n-6 PUFAs in the diet. Because both heterozygotes (, for example, C/T at APOA5−1131 locus) and homozygotes (for example, C/C at APOA5−1131 locus) are susceptible to increased plasma lipid, such as plasma TG levels when exposed to n-6 PUFAs, a kit must be able to detect at least the allele C or any allele in very tight linkage disequilibrium with, for example, allele C of the APOA5−1131 locus.

Accordingly, the invention provides a kit providing dietary advice to an individual comprising: a) a system for genotyping APOA5 locus for at least one polymorphic marker from a biological sample; and b) instructions that if one or two of alleles selected from the group consisting of APOA5−1131C, APOA5−3G, APOA5 IVS3+476A, and APOA5 1259C alleles is detected, the individual is advised to limit or avoid consumption of foods containing n-6 polyunsaturated fatty acids to avoid developing an atherogenic lipid profile, and that if no alleles selected from the group consisting of APOA5−1131C, APOA5−3G, APOA5 IVS3+476A, and APOA5 1259C or that the presence of a homozygous allele selected from the group consisting of APOA5−1131T, APOA5−3A, APOA5 IVS3+476G, and APOA5 1259T alleles is detected, the individual can consume n-6 polyunsaturated fatty acids without being at increased risk of developing an atherogenic lipid profile.

The instructions of the kit may also be given only in negative, i.e., that a person carrying one or more alleles selected from the group consisting of APOA5−1131C, APOA5−3G, APOA5 IVS3+476A, and APOA5 1259C, the individual is advised to avoid n-6 PUFAs. Alternatively, the instructions may be given in positive, i.e. individuals who do not have APOA5−1131C, APOA5−3G, APOA5 IVS3+476A, and APOA5 1259C alleles, can consume polyunsaturated fatty acids without being significantly predisposed to atherogenic plasma lipid profile, and may, in fact, benefit from consumption of PUFAs, including n-3 and n-6 PUFAs.

In one preferred embodiment, the kit comprises a plurality of oligonucleotide probes on a solid surface for detecting the polymorphisms in APOA5 locus. In one embodiment, the solid surface is a chip or a bead.

The invention also provides a kit for screening for individuals at risk of atherogenic plasma lipid profile when exposed to polyunsaturated fatty acids, wherein the kit comprises a plurality of isolated oligonucleotides, the oligonucleotides corresponding to no more than about 1, 2, 3, 4, 5, 6, 7, 8, 9, 10-20, 20-30, 30-40, 40-50, 50-60, 60-70, 70-80, 80-90, 90-100, 100-200, 200-300, 300-400, or up to about 500 nucleic acid polymorphisms, wherein at least one of the polymorphisms, alternatively at least two, alternatively at least three, alternatively at least 4, alternatively at least 5, 6, 7, or 8 of the polymorphisms is selected from the group consisting of APOA5−1131C/T, APOA5−3G/A, APOA5 IVS3+476A/G, and APOA5 1259C/T, and a guideline that indicates that if any of the polymorphic alleles consisting of APOA5−1131C, APOA5−3 G, APOA5 IVS3+476A, and APOA5 1259C is detected in a biological sample from an individual, the individual should be advised to avoid polyunsaturated fatty acids including n-3 and n-6 PUFAs, in one embodiment at least n-6 PUFAs.

The invention also provide a method for creating diet advice comprising providing a service, either by the service provider or by a third party provider to screen for at least one of the polymorphisms selected from the group consisting of APOA5−1131C/T, APOA5−3 G/A, APOA5 IVS3+476A/G, and APOA5 1259C/T in a biological sample from an individual. The method further comprises providing a service, either by a direct provider or by a third party provider to create a diet advice with restricted amount of polyunsaturated fatty acids for the individual whose sample contains at least one allele of APOA5−1131C, APOA5−3G, APOA5 IVS3+476A, or APOA5 1259C. The method further comprises delivering the diet advice to the individual. All the steps of the method can be performed by physically different entities from a biological sample taken from an individual who is in need of dietary advice to maintain or alter plasma lipid profile, wherein the goal is to achieve a less atherogenic lipid profile.

The dietary advice may contain recommendations to avoid n-6 PUFAs, which would include avoiding foods such as corn, safflower, sunflower, soybean, and cottonseed oils. The foods rich in n-3 PUFAs include, but are not limited to generally, marine/fish oils. Foods high in n-3 fatty acids include, but are not limited to salmon, halibut, sardines, albacore, trout, herring, walnut, flaxseed oil, and canola oil. Additionally, at least shrimp, clams, chunk light tuna, catfish, cod, and spinach contain n-3 PUFAs. Accordingly, the dietary advice may include lists of foods the consumption of which is preferred, such as those containing n-3 PUFAs, and lists of foods the consumption is to be avoided or limited, such as those containing n-6 PUFAs.

In addition, we found that the association between APOA5−1131T>C, −3A>G, IVS+476G>A and 1259T>C genetic variants and CCA IMT is modified by obesity related indicators. Three common haplotypes variants were formed by the five individual variants examined in this study. The haplotype defined by the substitution of G for C at nucleotide 56 was associated with higher CCA IMT compared to the wild type haplotype, whereas the haplotype defined by the presence of the rare allele in the −1131T>C, −3A>G, IVS+476G>A and 1259T>C genetic variants was associated with higher CCA IMT only in obese subjects.

Surprisingly, all of these associations were independent of cardiovascular risk factors including fasting triglycerides and other lipid levels. No significant associations were noted between APOA5 genetic variants and ICA IMT or carotid stenosis.

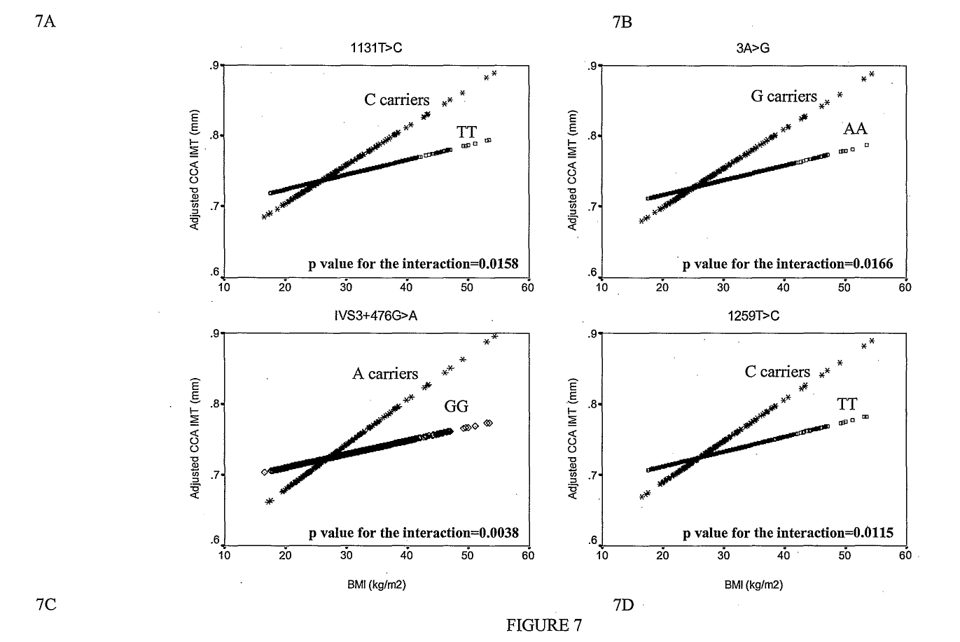

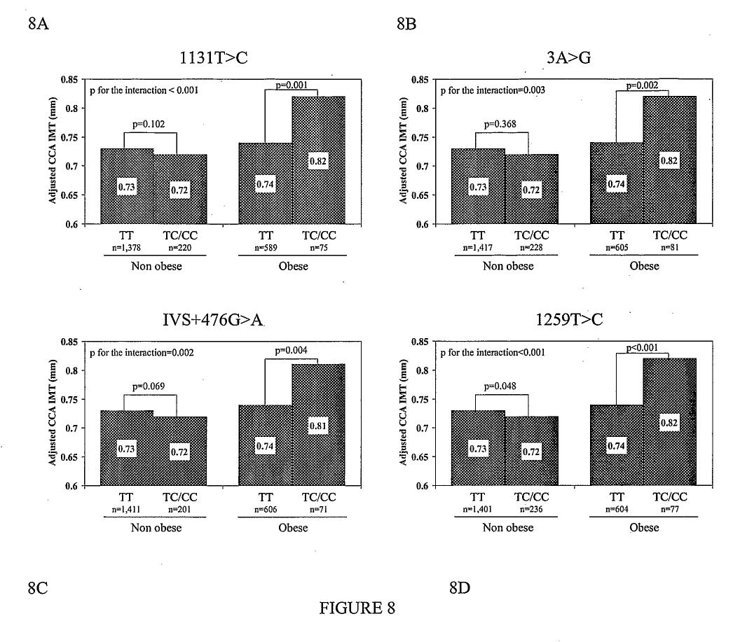

For example, FIGS. 7A-7D shows predicted values from the regression of the adjusted common carotid artery (CCA) intimal medial thickness on BMI according to the individual APOA5 genotypes, and p value for the interaction between these genotypes and BMI. Adjusted for age, sex, smoking, diabetes, systolic blood pressure, hypertension treatment, triglycerides, HDL and LDL cholesterol. Further, FIGS. 8A-8D shows multivariable adjusted common carotid artery intimal medial thickness (CCA IMT) according to the analyzed individual APOA5 genotypes and obesity, and p value for the interaction between these genotypes and obesity. Adjusted for age, sex, smoking, diabetes, systolic blood pressure, hypertension treatment, triglycerides, HDL and LDL cholesterol.

The results of our study extend and add new information regarding the association between APOA5 and atherosclerosis measures. Although the association between APOA5 genetic variants and hypertriglyceridemia has been extensively studied and consistently replicated in different studies (Pennacchio L. A., et al., 2001, Science. 294:169-73; Pennacchio L. A., et al., 2002, Hum Mol Genet. 11; 3031-8; Lai C. Q., et al., 2003, J Lipid Res. 44:2365-73), there have been few studies, particularly in large population-based cohorts, of the association between these genetic variants and clinically relevant atherosclerotic phenotypes (Lai C. Q., et al., 2004, J Lip Res. 45:2096-105; Talmud P. J., et al., 2004, J Lipid Res. 45:750-6; Szalai C., et al., 2004, Atherosclerosis. 173:109-14; Hubacek J. A., et al., 2004, Clin Genet. 65:126-30; Hsu L. A., et al., 2005, Atherosclerosis. July 26; [Epub ahead of print]). We report an association between APOA5 genetic variants and CCA IMT, and although the magnitude of differences in IMT is modest, similar magnitudes of difference in other studies have been associated with clinically significant atherosclerotic outcomes (Burke G. L., et al., 1995, Stroke. 26:386-91; O'Leary D. H., et al., 1999, N Engl J. Med. 340:14-22; Wang T. J., et al., 2003, Circulation. 108:572-6).

Our data further add to prior studies by analyzing the association between haplotypes as well as individual genetic variants at the APOA5 locus with atherosclerosis related phenotypes. While we did not observe statistically significant associations between the individual APOA5 variants and carotid measures, the haplotype defined by the presence of the 56C>G variant. (APOA5*1/3-3/3) was associated with higher CCA IMT compared to the wild type haplotype (APOA5*1/1). Thus, without wishing to be bound by a theory, the discordance of the results for CCA IMT observed between the association of the haplotype defined by the presence of the 56 C>G variant and this individual SNP may be explained by a more precise genetic classification of the individuals.

The haplotype analysis clearly defines three different haplotype-genotype groups. However, the individual SNP analysis defines only two groups, comparing the carriers of the 56 C>G variant vs non-carriers. Because the non-carriers includes both the wild-type individuals (haplotype APOA5*1/1) and the carriers of the other four genetic variants (haplotype APOA5*1/2-2/2) the association results for CCA IMT may be diluted by the introduction of misclassification. These results support the idea that analyses based upon haplotypes may be more efficient than separate analyses of individual markers when studying complex diseases (Morris R. W. and N. L. Kaplan, 2002, Genet Epidemiol. 23:221-33).

Of potential clinical relevance, we report a significant effect modification of obesity on the association of the individual APOA5 variants with CCA IMT. This effect modification was consistently noted in analyses of haplotypes, involving the APOA5*1/2-2/2 haplotype-genotype which was present in 11% of the population. These results suggest that the 56C>G acts as a restrictive variant, whereas the −1131T>C, −3A>G, IVS+476G>A and 1259T>C act as conditional genetic variants expressing the deleterious phenotype under certain environmental conditions, such as obesity. Interestingly, although all of the individual variants were associated with higher triglyceride levels, only the 56C>G variant was also associated with lower HDL-cholesterol whereas the −1131T>C, −3A>G and IVS+476G>A variants were also associated with higher total cholesterol. These different associations may be related to a different impact of these variants on apoAV function (Talmud P. J., et al., 2005, J Biol Chem. 2005 Jun. 7; [Epub ahead of print), whereas the APOA5 56C>G variant reduces APOA5 expression, the other four variants, that define the haplotype APOA5*2, do not influence translation efficiency or gene expression, suggesting that the effect of these variants may reflect a strong linkage disequilibrium with other functional variants. The APOA5 gene is a constituent of the well known APOA1/C3/A4/A5 gene cluster that has been the subject of intense research to gain understanding about lipid metabolism and cardiovascular disease risk (Lai C. Q., et al., 2005, Curr Opin Lipidol. 16:153-66). Recent data suggest that the haplotype APOA5*2 is in linkage disequilibrium with the minor alleles of some APOC3 variants (SstI −3238G>C-, −482C>T, and −455T>C) (Olivier M., et al., 2004, Genomics. 83:912-23; Talmud P. J., et al., 2002, Hum Mol Genet 11:3039-46), such that the association between this haplotype and carotid IMT in obese men and women could be explained by variations in the APOC3 gene. The APOC3 Sst1 variant has been associated with carotid atherosclerosis in different studies (Brown S. A., et al., 1993, Arterioscler Thromb 13; 1558-66; Pallaud C., et al., 2001, Clin Genet. 59:316-24; Islam M. J., et al., 2005, Atherosclerosis 180; 79-86) although we did not observe this association in the Framingham heart study (Elosua R., L. A. Cupples, C. S. Fox, J. F. Polak, R. A. D'Agostino Sr, P. A. Wolf, C. J. O'Donnell, J. M. Ordovas. Association between well-characterized lipoprotein-related genetic variants and carotid intimal medial thickness and stenosis: The Framingham Heart Study. Atherosclerosis, in press). Recent studies suggest that haplotypes in the APOC3 but not in the APOA5 gene increase susceptibility to myocardial infarction (Ruiz-Narvaez E. A., et al., 2005, J Lipid Res. 46:2605-13). Further studies are warranted to explore this cluster, define the LD pattern, and determine the association with lipid traits and cardiovascular risk susceptibility.

All of the associations of APOA5 variants with CCA IMT were independent of risk factors, including fasting triglycerides or other lipid levels. This surprising result has been also reported by Szalai et. al., who found that the association between APOA5−1131T>C variant and coronary heart disease susceptibility was independent of triglyceride levels (Szalai C., et al., 2004, Atherosclerosis. 173:109-14). These observations suggest additional mechanisms explaining the association between APOA5 and atherosclerosis independent of the classical lipid risk factors included in these analyses. It is possible that other lipid variables, such as postprandial lipemia or LDL particle size, previously reported to be associated with APOA5 genotype (Lai C. Q., et al., 2004, J Lip Res. 45:2096-105; Austin M. A., et al., 2004, Biochem Biophysica Acta. 1688:1-9), may play a role. We explored other possible mechanisms by including CRP, a marker of inflammation, and the ratio between triglycerides and HDL-cholesterol, as an indirect marker of insulin resistance, in the multivariate models; however, the inclusion of these covariates did not alter the reported associations.

We observed an association of the APOA5 variants with CCA IMT but not with ICA IMT or stenosis. These different carotid phenotypes represent different stages in the complex process of atherosclerosis (Spence J. D. and R. A. Hegele. 2004, Stroke. 35:649-53). The observation of associations specific to CCA but not ICA, the effect modification by obesity, and the independence of effect from circulating triglyceride and other lipid levels, leads to speculation regarding the function of apoAV in atherogenesis. ApoAV has been shown to stimulate lipoprotein lipase hydrolytic capacity, (Schaap F. G., et al., 2004, J Biol Chem. 279:27941-7; Fruchart-Najib J., et al., 2004, Biochem Biophys Res Commun. 319:397-404) and it is possible that it also stimulates other lipoprotein lipase functions such as the lipoprotein bridging and selective cholesteryl ester uptake (Stein Y. and O. Stein. 2003, Atherosclerosis. 170:1-9). These functions are important in the delivery of atherogenic particles to the arterial wall (Stein Y. and O. Stein. 2003, Atherosclerosis. 170:1-9) and could be especially significant in arterial segments with laminar blood flow, such as the CCA, where the wall shear stress decreases (Gnasso A., et al., 1996, Circulation. 94:3257-62).

In this study, we focused on the APOA5 variants that have been recently defined and intensively studied as well as on carotid phenotypes that are both heritable (Fox C. S.; et al., 2003, Stroke. 34:397-401) and independently associated with incident cardiovascular disease in prospective studies (O'Leary D. H., et al., 1999, N Engl J. Med. 340:14-22).

In addition, our present findings apply to a Caucasian cohort.

We showed that common APOA5 genetic variants and haplotypes are associated with CCA IMT particularly in obese subjects. These associations have substantial public health implications, given the recent epidemic increases in obesity. The independence of these associations from fasting triglyceride levels suggests the existence of an alternative mechanism in the association between APOA5 and atherosclerosis. Further studies are certainly warranted to confirm and further explore these findings.

Accordingly, the invention also provides a method of identifying individuals with higher risk for developing CCA IMT by analyzing at least one locus selected from −1131T>C, −3A>G, IVS3+476G>A, and 1259T>C for the rare allele, wherein the presence of the rare allele is indicative of the individual being at higher risk, particularly if the individual is obese, of developing CCA IMT. In one embodiment, one analyzes all four of these loci. In one embodiment, one uses loci in tight linkage disequilibrium with the alleles in said loci.

Accordingly, in one embodiment, the method for personalized diet design further comprises a step of determining whether one or more of the rare alleles in the loci −1131T>C, −3A>G, IVS3+476G>A, and 1259T>C are present and if so, advising the individual carrying such allele or a haplotype comprising such allele to particularly monitor their weight and keep it under the overweight or at least obese levels in order to decrease their risk for CCA IMT and associated diseases, such as atherosclerosis.

The references cited herein and throughout the specification are herein incorporated by reference in their entirety.

### EXAMPLES

### Example 1

Apolipoprotein A5 gene (APOA5) variation is associated with increased levels of plasma triglycerides (TG). However, little is known about how dietary fat modulates the effect of APOA5 variation on TG-rich lipoprotein metabolism (TRL).

Methods and Results—We investigated the interaction between APOA5 gene variation and dietary fat in determining TRL metabolism, focusing on remnant-like particle (RLP) concentrations and lipoprotein particle size, in 1001 men and 1147 women participating in the Framingham Heart Study. Two polymorphisms, −1131T>C and C56>G, representing two independent haplotypes, were analyzed. Statistically significant gene-diet interactions between the −1131T>C polymorphism and polyunsaturated fat (PUFA) intake were found (p<0.001) in determining RLP concentrations and particle size, but not for the C56>G polymorphism. The −1131C allele was significantly associated with higher TG and RLP concentrations (P<0.01) only in the subjects consuming a high PUFA diet (6% or more of total energy). No heterogeneity by gender was found. These interactions also showed a clear dose-response effect when PUFA intake was considered as a continuous variable (P<0.01). Similar gene-nutrient interactions were found for the sizes of very-low density lipoprotein (VLDL) and low-density lipoprotein (LDL) particles. Only in carriers of the −1131C allele did the size of these particles increase (VLDL) or decrease (LDL) as PUFA intake increased (P<0.01). We further analyzed the effects of n-6 and n-3 fatty acids and found that the PUFA-APOA5 interactions were specific for dietary n-6.

Conclusions—Higher n-6 PUFA intake (but not n-3) significantly increased TG and RLP concentrations and VLDL size, and decreased LDL size in carriers of the APOA5−1131C allele. These results suggest that a diet high in n-6 PUFA is related to a more atherogenic lipid profile in these subjects.

Postprandial lipemia, characterized by a rise in triglyceride (TG)-rich lipoproteins (TRL) after eating1 has been the focus of numerous studies since Zilversmith2 in the late seventies proposed an important role of the postprandial state in atherogenesis. However, one of the major obstacles to gaining a deeper understanding of the role of remnant lipoproteins as cardiovascular disease (CVD) risk factors has been the technical complexity associated with their isolation and measurement in large population studies. In addition, current evidence suggests that genetic factors are significant determinants of the dramatic interindividual differences in plasma lipoprotein responses to a fat challenge3 and consequently to the variability in remnant lipoprotein concentrations. The quantitation of these particles has recently been facilitated by an assay based on the immunoseparation of remnant-like particles (RLP) and measurement of their cholesterol (RLP-C) and triglyceride (RLP-TG) contents.4 Using this approach, it has been shown that plasma RLP-C and RLP-TG concentrations measured in the fasting state are a reasonable surrogate of the concentration of lipoprotein remnants during the postprandial state.4, 5 Moreover, RLP has been shown to be an independent CVD risk factor in several studies, 4, 6, 7, 8. However, the contribution of genetic and dietary factors to remnant lipoproteins and CVD risk remains to be investigated.

We investigated whether dietary PUFA (total, n-6 and n-3) could modulate the effect of APOA5 variants on plasma TRL metabolism (by focusing on RLPs concentrations and VLDL and LDL particle size) in a large population-based study.

Study Design and Subjects: The study sample consisted of 2148 subjects who participated in the Framingham Offspring Study (FOS). Detailed design and methodology for the FOS has been described previously26. Fasting venous blood samples were collected and plasma was separated from blood cells by centrifugation and immediately used for the measurement of lipids. Lipids, CVD risk factors, and dietary intake were recorded for subjects who participated in the fifth examination visit conducted between 1992 and 1995 (n=3515). In addition, blood samples for DNA isolation were obtained from each subject during 1987-1991. The Institutional Review Board for Human Research at Boston University and the Human Investigation Research Committee at Tufts University/New England Medical Center approved the protocol of the study reported here. All participants provided written informed consent, underwent standardized clinical examination and provided fasting blood samples.

Only subjects with phenotypic data and complete dietary information for whom the APOA5 gene variation was examined, were included in this study. In addition, subjects taking lipid-lowering medications, as well as subjects with any missing data regarding control variables (age, BMI, smoking, alcohol consumption, diabetes status, β-blocker use, diuretic use, and estrogen use in women) were excluded from our analyses. Thus, 1001 men and 1147 women who fulfill the above criteria were analyzed. Alcohol consumption was calculated based on the reported alcoholic beverages consumed in the previous year for each individual, and subjects were classified as nondrinkers (those who did not report consumption of alcohol), and drinkers. Smokers were defined as those who smoked at least 1 cigarette/day.

Genetic analysis: Genomic DNA was isolated from peripheral blood leukocytes by standard methods. APOA5 SNPs −1131T>C (rs662799), −3A>G (rs651821), IVS3+476G>A (rs2072560), and 1259T>C (rs2266788) and 56C>G (rs3135506) were genotyped as previously described.19

Measurement of Plasma Lipid, Lipoprotein, and Apolipoprotein: The standardized procedures for sample collection and biochemical analysis for TG, total cholesterol, low-density lipoprotein cholesterol (LDL-C) and HDL-C have been described.27 VLDL and LDL subclass distributions were determined by proton nuclear magnetic resonance (NMR) spectroscopy.28 Measurements of RLP-TG and RLP-C concentrations were previously described.4 In brief, RLPs were separated by mixing 5 μL plasma with 300 μL immunoseparation gel consisting of monoclonal antibodies to apo B-100 and apo A-I. After 2 h of incubation at room temperature, cholesterol and TGs in the unbound fraction were measured by sensitive cholesterol and TG assays.

Dietary Assessment Dietary intake was estimated with the semi-quantitative Willett food-frequency questionnaire (136 food items) with specified serving sizes29, 30. The Harvard University Food Composition Database derived from US Department of Agriculture sources and supplemented with manufacturer information was used to calculate nutrients. This questionnaire was validated to estimate total PUFA intake as well as n-3 and n-6 fatty acids.29, 30 Fat intake data were obtained in terms of absolute amounts (g/day). The effect of fat in terms of nutrient density was then modeled, i.e., the ratio of energy from fat to total energy, expressed as a percentage. Intakes of total fat, saturated fatty acids (SFA), monounsaturated fatty acids (MUFA), total PUFA, n-3 and n-6 were calculated for each individual. These were included in analyses as both continuous and categorical variables. To construct categorical variables, intakes were classified into two groups according to the mean value of the population (i.e., one group had intakes below the mean and one group had intakes above it). In addition, we considered together as n-3 the dietary intake of alpha linolenic acid (ALA), eicosapentaenoic acid (EPA), docosahexaenoic acid (DHA) and docosapentaenoic acid (DPA). Linoleic acid (LA) and arachidonic acid were considered together as n-6 PUFA. Tertiles of PUFA intakes (n-6 and n-3) were also considered.

Statistical Analyses We examined all continuous variables for normality of distribution. TG and RLP-TG and RLP-TC concentrations were log transformed. The relation between APOA5 genotypes, dietary PUFA, and plasma lipid-related measures was evaluated by analysis of covariance techniques. Because our study involved some correlated data due to familial relations (siblings and cousins), a generalized linear mixed model approach to adjust for this correlation was employed. An exchangeable correlation structure was assumed and the analysis using the Mixed procedure in SAS was performed. The interactions between dietary PUFA (as a continuous or as a categorical variable) and the APOA5 polymorphisms were tested in a hierarchical multivariate interaction model after controlling for potential confounders including gender, age, BMI, smoking, alcohol consumption, diabetes status, β-blocker use, diuretic use, estrogen use (in women), energy intake. These analyses were performed for the whole sample and for men and women separately in order to verify the homogeneity and the magnitude of the effect. Standard regression diagnostic procedures were used to ensure the appropriateness of these models. When PUFA intake was considered to be a continuous variable, its interaction with the APOA5 polymorphism was depicted by computing the predicted values for each individual from the adjusted regression model and plotting these values against PUFA intake depending on the APOA5 genotype. All reported statistical tests were two-sided. Statistical analysis was carried out using SAS software. To minimize Type I errors that are increased due to the high number of comparisons, only P values≦0.01 were considered statistically significant.

RESULTS: Information regarding demographic, biochemical, dietary intake, and genotypic data according to gender is provided in Table 1. Genotype frequencies did not deviate from Hardy-Weinberg equilibrium expectations. As previously shown, four of the APOA5 SNPs (−1131T>C, −3A>G, IVS3+476G>A, and 1259T>C) were in strong LD with each other and shared the same haplotype (i.e., APOA5*2); in contrast, SNP 56C>G is independent of the aforementioned polymorphisms, representing a different haplotype (i.e., APOA5*3) (15, 19). We investigated gene-diet interactions determining plasma TG, TRL concentrations and lipoprotein particle sizes for each of the five SNPs −1131T>C, −3A>G, 56C>G, IVS3+476G>A, and 1259T>C. As expected, the analyses of each of the four SNPs in strong LD revealed similar interactions with the dietary fat intake on the variables examined; whereas no significant interactions were observed for SNP 56C>G (data not shown). The results are presented for SNP −1131T>C.

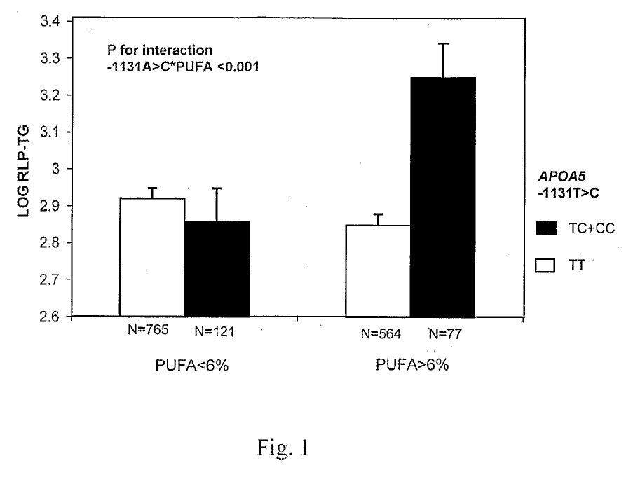

To examine the interaction between the −1113T>C SNP and dietary fat, we first dichotomized dietary variables according to the population mean. Homozygotes (CC) and heterozygotes (TC) of the rare allele were combined to increase statistical power. Separate models were fitted for men and women and, although the magnitude of the effect was slightly greater in men, similar direction of the effects was observed. Moreover, the homogeneity by gender was also confirmed in examining the P value of the corresponding interaction term (P>0.05). Therefore, men and women were analyzed together. Statistical significant interactions p<0.001) between the SNP −1131T>C and PUFA intake (more than or less than 6% of energy) on TG concentration were found. In the adjusted model, the C allele was associated with an increase in TG only in subjects consuming more than 6% of energy from PUFA. However, mean TG concentrations were not higher in carriers of the C allele as compared with the TT homozygotes when the PUFA consumption was low. We also observed similar and significant interactions (P<0.001) between PUFA consumption and SNP −1131T>C on RLP-C and RLP-TG (FIG. 1). When we analyzed the interaction of SNP −1131T>C and PUFA intake in determining LDL and VLDL sizes, we also found statistically significant interactions that were consistent with a more atherogenic lipid profile in subjects carrying the C allele and consuming more than 6% of energy from PUFA. Thus, in these subjects a high PUFA intake was associated with a decrease of LDL particle size as well as with increased VLDL size. The interaction effects between APOA5−1131T>C SNP and PUFA intake remained statistically significant even after additional adjustment for total fat intake. Using the same statistical models did not uncover any significant interactions between the APOA5−1131T>C SNP and the intake of total fat, SFA or MUFA on TG, RLP-TG, RLP-C concentrations or LDL and VLDL particle size.

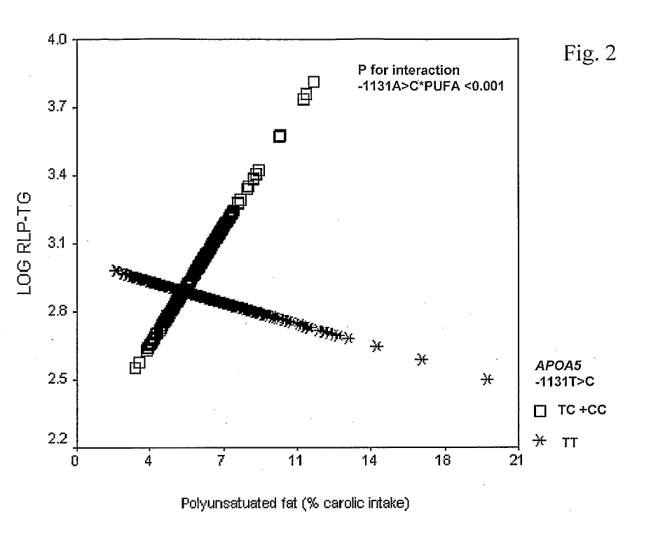

To investigate whether the interaction between PUFA and APOA5−1131T>C was dose-dependent, we analyzed fat intake as a continuous variable. To show the homogeneity by gender, separate models for men and women were fitted. Table 2 shows the statistical significance of the interaction term between fat intake (total fat, SFA, MUFA and PUFA) and the APOA5 polymorphism in determining TG, RLP-TG, RLP-C, LDL and VLDL particle size by gender after controlling for potential confounders. Consistent with the results of the categorical variables, we found a significant interaction between PUFA intake and −1131T>C SNP on TG, RLP-TG, RLP-C, and VLDL size in both men (P<0.001, P=0.002, P<0.001 and P=0.002, respectively) and women (P<0.001, P<0.001, P=0.005 and P=0.002, respectively). Although the interaction term for LDL particle size was statistically significant only in men, the direction of the effect was similar in women, and no significant heterogeneity was found. In the combined analysis by gender, all these interaction terms with PUFA intake were significant. As an example to illustrate these modulations, FIG. 2 shows the modification of the effect of the SNP −1131T>C by PUFA intake on RLP-TG concentrations in men and women combined. Differences in slope of the regression lines indicate that the effect of the −1131T>C on RLP-TG depends on the amount of PUFA consumed. High PUFA intake was associated with lower RLP-TG concentrations in TT individuals whereas increased PUFA intake increased RLP-TG concentrations in carriers of the C allele.

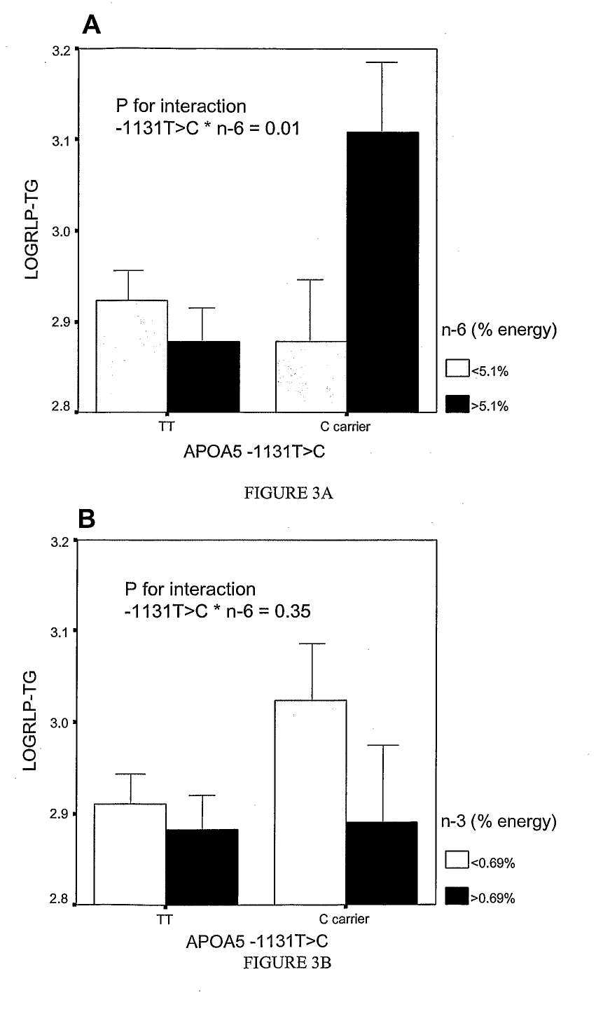

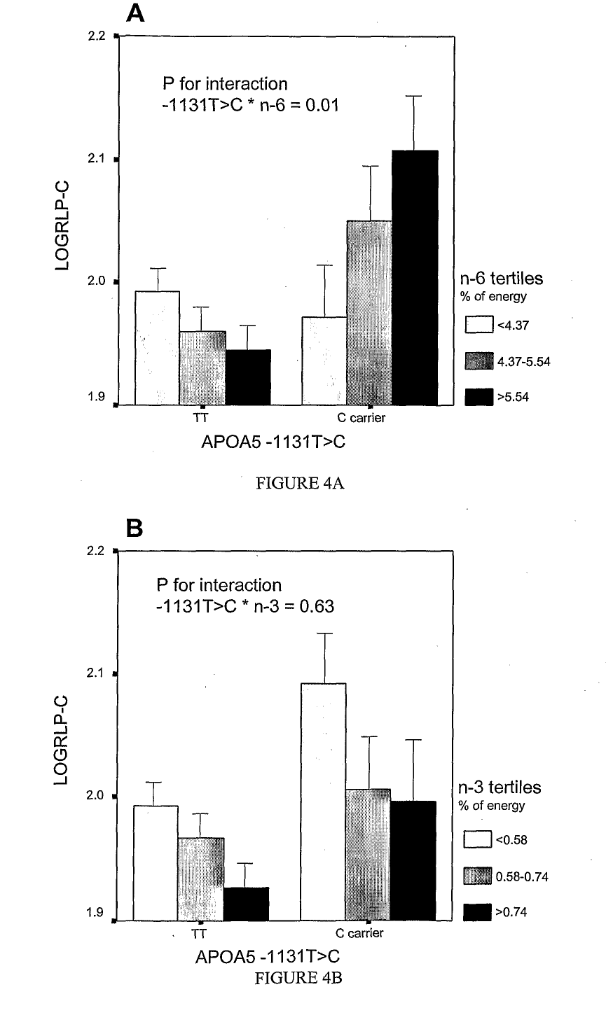

To uncover the constituents of dietary PUFA responsible for these findings, the individual contributions of each PUFA family—either n-3 or n-6 PUFA—to the above reported interactions were examined. For this purpose, n-6 and n-3 fatty acid intakes were taken as two categorical variables, according to the population mean (5.10% of energy for n-6 and 0.69% of energy for n-3). As no heterogeneity of the effects by gender was detected, data for men and women were analyzed together. After adjustment for covariates (gender, age, familial relationships, BMI, smoking, alcohol, diabetes status, beta blocker, estrogens and energy), statistically significant interaction effects were found for n-6 fatty acid intake and the −1131T>C polymorphism in determining plasma RLP-TG (FIG. 3A). No significant interactions were observed between the −1131T>C and n-3 PUFA on RLP-TG (FIG. 3B). Results for RLP-TC, TG and lipoprotein particle size were similar. To explore a possible dose-response relationship in this interaction, n-6 and n-3 intake were considered as population tertiles (<4.37% of energy, 4.37%-5.54% of energy, and >5.54% of energy for n-6 and <0.58% of energy, 0.58%-0.74% of energy, and >0.74% of energy for n-3). We observed significant interactions between −1131T>C and n-6 PUFA intake on RLP-TC concentrations (FIG. 4A), RLP-TG and TG with a clear dosage effect. However, no significant interactions between the −1131T>C polymorphism and n-3 PUFA in determining RLP-TC concentrations (FIG. 4B), RLP-TG and TG were found, suggesting that the global effect previously observed for total PUFA is specific to consumption of n-6 PUFAs.

DISCUSSION: In this study, we report a significant and consistent interaction between a specific variant of APOA5, −1131T>C (representing haplotype APOA5*2), and PUFA dietary intake on the concentration of plasma TG, RLP-TG, RLP-C, as well as VLDL and LDL sizes in the Framingham Heart Study. When PUFA consumption was high (specifically above the population mean, or 6% of total energy intake), carriers of the −1131C allele exhibited significantly higher concentrations of remnant lipoproteins (RLP-TG and RLP-C). Moreover, carriers of the −1131C allele displayed on average larger VLDL and smaller LDL molecules as compared with TT individuals. Larger VLDL and smaller LDL particles have been reported to increase CVD risk.32 This interaction revealed a clear dose-dependent effect as well as a biologically plausible association consistent with the expected metabolic pathways involved. Moreover, two additional findings in this study add interest to this observation. First, the interaction observed for the SNP −1131T>C was not shared by the SNP 56C>G, which represents the other common APOA5*3 haplotype, despite the fact that we reported similar associations between these two haplotypes and plasma TRL concentrations.19 The contrasting results suggest that the hypertriglyceridemic phenotype associated with these two haplotypes is driven by different mechanisms. The causative mutation for the APOA5*2 haplotype is associated with dietary response, whereas that of the APOA5*3 (56C>G) haplotype may not be modulated by dietary factors. Second, the reported interactions are exclusively due to consumption of PUFA, with no interactions detected for total fat, SFA and MUFA.

Since n-3 and n-6 fatty acids result in metabolic products differing from each other in terms of their potential preventive effect on CVD,33 we further investigated whether the PUFA interaction applied to the consumption of both families of PUFA. Our results support the notion that the above reported interactions are specific to n-6 PUFA. The potentially negative effects associated with elevated lipoprotein remnant concentrations observed in carriers of the APOA5−1131C allele who consume high PUFA n-6 were not observed for the consumption of PUFA n-3. In fact, a protective effect of n-3 fatty acids was observed regardless of genotype. This observation supports the differential role observed for these two families of PUFA on the dietary prevention of CVD33 consistent with findings observed for other loci. For example, promoter variants of arachidonate 5-lipoxygenase (ALOX5) are associated with increased atherosclerosis and this association is promoted by dietary n-6 PUFA and inhibited by marine n-3 PUFA34.

The relevance of these findings for CVD risk detection and prevention lies on the correlation between RLP levels and postprandial lipemia, a potential CVD risk factor.5, 35 In the mouse model, Fruchart-Najib et al 13 have reported that overexpression of hAPOA5 diminishes postprandial TG response to sunflower oil (n-6 PUFA) loading. Therefore, one may hypothesize that the observed differences between carriers of different alleles at the APOA5−1131T>C may be due to their differential expression. However, recent findings do not support this notion36. Still, based on the correlation between fasting and postprandial TG concentrations, we anticipate that the APOA5−1131C minor allele will result in higher postprandial lipemia as compared with TT homozygotes. In this regard, Jang et al 37 demonstrated that male carriers of the −1131C allele had higher chylomicron-TG area under curve than homozygotes for the −1131T allele after a fat load enriched in PUFA (mainly n-6). Conversely, the allelic differences were not significant when subjects were provided with a low fat load37. Martina et al38 also reported that male carriers of the −1131C allele had significantly higher lipemic responses to a fat tolerance test. However, the responses were not significantly different by genotype after adjustment for fasting TG levels. An apparent paradoxical observation was reported by Masana et al39 in which carriers of the C allele had similar or even lower incremental diurnal triglyceridemia than wild type carriers after correction for fasting capillary TG levels. We speculate that the high saturated fat meal used for the fat load used by Masana et al39 prevented the manifestation of the increased postprandial load, which appears to be preferentially driven by PUFA n-6.

The importance of these findings to the notion of personalized dietary recommendations is noteworthy. The frequency of the −1131C allele carriers ranges from 0.13 in Whites, to 0.20 in Africans, 0.30 in Hispanics and up to 0.40-0.50 in Chinese and Japanese populations. Assuming this interaction between the −1131C variant of APOA5 and PUFA dietary intake on the concentration of plasma TG, RLP-TG, RLP-C, as well as VLDL and LDL sizes, holds across ethnic boundaries we hypothesize that the APOA5−1131C allele may have a contribution to CVD risk in Asian populations, especially in the context of increased fat consumption observed in recent years. It is interesting to note that most dietary intervention studies using PUFA-rich diets have been performed in Whites, where frequencies of the high-TG-associated APOA5 alleles are relatively low. Hence, the overall CVD risk associated with the −1131C allele in the presence of high PUFA n-6 diets may not be apparent for the population as a whole. In addition, although the current ratio of n-6 and n-3 PUFA in the typical North American diet was estimated in the range of 10:1 to 25:1; 33 All these aspects should be carefully considered before making general PUFA intake recommendations.

In summary, the observed interaction between APOA5 and PUFA consumption on TG, RLP concentrations and lipoprotein particle sizes indicates that in carriers of the −1131C allele a higher n-6 PUFA intake increases plasma TG and RLP as well as decreases LDL particle size, contributing to a more atherogenic lipid profile. This interaction is specific to n-6 PUFA and it is observed for the haplotype identified using the −1131T>C polymorphism. These data are consistent with the notion that high PUFA diets may not be the best dietary recommendation for everyone with respect to CVD risk, and that increased emphasis should be made regarding the balance between n-3 and n-6 fatty acids. However, these observational findings require further replication in other populations as well as in dietary intervention studies.

TABLE 1
Demographic, Biochemical, Dietary, and Genotypic
Characteristics of Participants According to Sex

| | Men | | Women | |
|---|---|---|---|---|
| | (n = 1,001) | | (n = 1,147) | |
| Age, years | 50.8 | (9.9) | 50.1 | (9.6) |
| BMI, kg/m2 | 27.6 | (3.8) | 25.9 | (5.5) |
| LDL-C, mg/dl | 135 | (33) | 127 | (35) |
| HDL-C, mg/dl | 44 | (11) | 56 | (15)* |
| TG, mg/dl | 135 | (97) | 105 | (101)* |
| RLP-TG†, mg/dl | 30.7 | (45.0) | 22.2 | (70.2)* |
| RLP-C†, mg/dl | 8.54 | (6.79) | 7.13 | (6.31)* |
| LDL diameter, nm | 20.69 | (0.59) | 21.08 | (0.46) |
| VLDL diameter, nm | 48.28 | (9.24) | 44.20 | (8.58)* |
| Glucose, mg/dL | 97.0 | (23.0) | 91.1 | (19.3)* |
| Energy intake, Kcal/d | 2004 | (643) | 1745 | (576)* |
| Total fat, g/d | 67.1 | (27.6) | 56.9 | (23.8)* |
| SFA, % of energy | 10.6 | (2.9) | 10.3 | (2.9)* |
| MUFA, % of energy | 11.5 | (2.6) | 11.0 | (2.6)* |
| n-6 PUFA, % of energy | 5.1 | (1.5) | 5.2 | (1.6) |
| n-3 PUFA, % of energy | 0.65 | (0.2) | 0.71 | (0.2)* |
| PUFA, % of energy | 5.8 | (1.6) | 6.0 | (1.7) |
| Animal fat, % of energy | 15.9 | (5.5) | 15.1 | (5.1)* |
| Vegetable fat, % of energy | 14.0 | (4.4) | 14.1 | (4.5) |
| Carbohydrate, % of energy | 50.2 | (8.5) | 51.9 | (8.4)* |
| Fiber, g/d | 19.1 | (8.2) | 19.2 | (8.4) |
| Alcohol, g/day | 14.7 | (19.4) | 7.2 | (11.8)* |
| Drinkers, n (%) | 751 | (75) | 768 | (67)* |
| Smokers, n (%) | 233 | (23.3) | 247 | (21.6) |
| on beta-blocker treatment, n (%) | 111 | (11.1) | 73 | (6.3)* |
| Diabetes status, n (%) | 74 | (7.4) | 44 | (3.8) |
| on estrogen treatment, n (%) | 0 | (0) | 90 | (7.9) |

APO45 −1131T > C‡, n (%)

| | | | | |
|---|---|---|---|---|
| TT | 816 | (86.2) | 936 | (87.0) |
| C carriers | 131 | (13.8) | 140 | (13.0) |

APOA5 56C > G‡, n (%)

| | | | | |
|---|---|---|---|---|
| CC | 860 | (87.5) | 1002 | (89.1) |
| G carriers | 123 | (12.5) | 123 | (11.0) |

Values are listed as mean (SD) or n (%)
*Significantly different from men (P < 0.05).
†Data for these variables were only available in a random sample of these participants (n = 1600).
‡APOA5 genetic data was successfully obtained in 947 men and 1076 women for the −1131T > C polymorphism and for 983 men and 1125 women for the 56C > G polymorphism.
BMI: Body mass index, LDL-C: Low-density lipoprotein cholesterol, HDL-C: High density lipoprotein cholesterol, TG: triglycerides, RLP-TG: remnant like particles triglycerides, RLP-C: remnant like particles cholesterol.

TABLE 2
Interaction of fat consumption with APOA5 −1131T > C genotypes on TG-rich particle concentration and
the size of LDL and VLDL in the Framingham Heart Study by gender. P values and regression coefficients

| | | Total Fat | | | SFA | | | MUFA | | | PUFA | | |
|---|---|---|---|---|---|---|---|---|---|---|---|---|---|
| | | B (SE) | | P | B (SE) | | P | B (SE) | | P | B (SE) | | P |
| TG | M | −0.003 | (0.013) | 0.806 | −0.038 | (0.028) | 0.178 | −0.006 | (0.030) | 0.834 | 0.142 | (0.037) | <0.001 |
| | F | 0.019 | (0.012) | 0.108 | 0.005 | (0.018) | 0.787 | 0.025 | (0.026) | 0.338 | 0.109 | (0.027) | <0.001 |
| RLP-TG | M | 0.005 | (0.019) | 0.805 | −0.063 | (0.035) | 0.074 | 0.036 | (0.044) | 0.423 | 0.205 | (0.068) | 0.0023 |
| | F | 0.027 | (0.017) | 0.116 | 0.010 | (0.024) | 0.664 | 0.031 | (0.038) | 0.417 | 0.151 | (0.042) | <0.001 |
| RLP-C | M | 0.002 | (0.011) | 0.883 | −0.046 | (0.020) | 0.022 | 0.015 | (0.024) | 0.526 | 0.160 | (0.044) | <0.001 |
| | F | 0.011 | (0.010) | 0.271 | −0.003 | (0.013) | 0.828 | 0.008 | (0.022) | 0.697 | 0.077 | (0.027) | 0.005 |
| LDLSZ | M | −0.022 | (0.010) | 0.032 | −0.024 | (0.022) | 0.280 | −0.047 | (0.026) | 0.071 | −0.154 | (0.032) | <0.001 |
| | F | −0.008 | (0.010) | 0.460 | 0.001 | (0.018) | 0.943 | −0.010 | (0.023) | 0.658 | −0.038 | (0.024) | 0.120 |
| VLDLSZ | M | 0.099 | (0.173) | 0.567 | −0.493 | (0.361) | 0.173 | 0.409 | (0.417) | 0.327 | 2.261 | (0.692) | 0.002 |
| | F | 0.409 | (0.196) | 0.037 | 0.264 | (0.304) | 0.387 | 0.912 | (0.424) | 0.032 | 1.671 | (0.529) | 0.002 |

TG: Triglycerides, RLP-TG: remnant-like particle TG, RLP-C: remnant like particle cholesterol, LDLSZ: low-density lipoprotein particle size, VLDLSZ: very-low-density particle size, SFA: saturated fatty acids, MUFA: monounsaturated fatty acids, PUFA: polyunsaturated fatty acids. M: male, F: female. Five separate regression models for each type of fat and gender were fitted. B indicates regression coefficient (mg/dL); SE, standard error B. P: statistical significance of the interaction term in the corresponding multivariate adjusted model. Models were adjusted for age, body mass index, familial relationships, smoking, alcohol, estrogens, diabetes, β-blockers, and energy intake. Fat intake was considered as a continuous variable. APOA5−1131T>C polymorphism was included as a two categories variable (TT versus C carriers) being the TT genotype as the reference category.

### Example 2

Genetic variation at the apolipoprotein A5 (APOA5) locus is associated with elevated triglyceride concentrations, a risk factor for atherosclerosis. Carotid intimal medial thickness (IMT) is a surrogate measure of atherosclerosis burden. We sought to determine the association of common APOA5 genetic variants with carotid IMT and stenosis.

In this study, 2,273 Framingham offspring study participants underwent carotid ultrasound and had data on at least one of the five APOA5 variants (−1131T>C, −3A>G, 56C>G, IVS3+476G>A and 1259T>C). Although none of the individual variants were significantly associated with carotid measures, the haplotype defined by the presence of the rare allele of the 56C>G variant was associated with a higher common carotid artery (CCA) IMT compared to the wild-type haplotype (0.75 mm vs 0.73 mm, p<0.05). The rare allele of each of the −1131T>C, −3A>G, IVS3+476G>A and 1259T>C variants and the haplotype defined by the presence of the rare alleles in these four variants were each significantly associated with CCA IMT in obese participants. These associations remained significant even after adjustment for triglycerides.

We found that APOA5 variants were associated with CCA IMT, particularly in obese participants. The mechanism of these associations and the effect modification by obesity is independent of fasting triglyceride levels.

Although hypertriglyceridemia is an independent risk factor for coronary heart disease (Hokanson J. E. and M. A. Austin, 1996, J Cardiovasc Risk. 3:213-9), there are limited data regarding the association between the APOA5 locus and a phenotypic manifestation of atherosclerosis in humans, particularly in unselected men and women from the general population. Most of the available studies have reported associations of APOA5 with atherosclerosis progression in post-coronary-bypass men (Talmud P. J., et al., 2004, J Lipid Res. 45:750-6), with coronary heart disease in a selected population of patients referred for coronary bypass surgery (Szalai C., et al., 2004, Atherosclerosis. 173:109-14) or for angiography (Bi N., et al., 2004, Mol Genet Metab. 83:280-6), and with myocardial infarction (Hubacek J. A., et al., 2004, Clin Genet. 65:126-30; Hsu L. A., et al., 2005, Atherosclerosis. July 26; [Epub ahead of print]) and cardiovascular disease (Lai C. Q., et al., 2004, J Lip Res. 45:2096-105) in the general population.

Carotid intima-media thickness (IMT) measured by ultrasound is associated with prevalent cardiovascular disease (Burke G. L., et al., 1995, Stroke. 26:386-91), incident myocardial infarction and stroke (O'Leary D. H., et al., 1999, N Engl J Med. 340:14-22) and premature parental coronary heart disease (Wang T. J., et al., 2003, Circulation. 108:572-6). Therefore, carotid IMT, as well as carotid stenosis, is widely used as a surrogate measure of atherosclerosis burden and risk. There is a substantial heritable component to both internal and common carotid IMT (Fox C. S., et al., 2003, Stroke. 34:397-401), but each may represent distinct underlying pathophysiologies (Gnasso A., et al., 1996, Circulation. 94:3257-62).

In this study, we sought to determine the association of APOA5 genetic variants and APOA5 haplotypes with carotid IMT and stenosis, to assess whether other cardiovascular risk factors modify this association, and to establish whether any observed associations are mediated through plasma triglycerides concentration, in a large community-based sample of men and women.

Study population: The design of the Framingham Heart Study has been previously detailed (26). Subjects included in this analysis were participants in the Offspring cohort of the Framingham Heart Study. There were 3,532 participants in Offspring Study examination cycle 6 (1995 to 1998). A total of 3,380 (96%) of these participants underwent B-mode carotid ultrasonography. APOA5 genotype data were available in 2,273 to 2,367 participants (67-70%) with available carotid IMT data depending on the genetic variant analyzed. The research protocol and genotype analyses were approved by the Institutional Review Boards at Boston University and Tufts University. All participants provided informed consent.

Carotid Ultrasonography Ultrasound measures were acquired and images analyzed according to a standard protocol (Polak J. F., et al., 1993, Radiology. 188:363-70) as has been previously described (Wang T. J., et al., 2002, Arterioscler Thromb Vasc Biol. 22:1662-7). Common carotid artery (CCA) and internal carotid artery (ICA) IMT were defined as the mean of the maximal IMT measurements for the right and left sides. A subjective estimate of ICA narrowing, graded as 0%, 1 to 24%, 25 to 49%, was made by the sonographer when Doppler-derived peak systolic velocities in the ICA were <150 cm/s. ICA narrowing of hemodynamic significance (≧50%) was defined as present when peak-systolic velocities in the ICA were ≧150 cm/s. We defined the degree of stenosis based upon the maximum stenosis in either ICA, and the stenosis was defined as present if it was ≧25%.

Apolipoprotein A5 genotype: DNA was isolated from blood samples using DNA blood Midi Kits (Qiagen, Hilden, Germany) following the protocol recommended by the vendor. Five previously reported variants were determined (Pennacchio L. A., et al., 2001, Science. 294:169-73; Pennacchio L. A., et al., 2002, Hum Mol Genet. 11; 3031-8): −1131T>C (initially named SNP3-[1]-), −3A>G, 56C>G (also known as S19W-[6]-), IVS3+476G>A (initially named SNP2-[1]-) and 1259T>C (also known as SNP1-[1]-).

Genotyping was carried out using the ABI Prism SnaPshot multiplex system (Applied Biosystem, Foster City, Calif.). The primers and probes used have been previously reported (Lai C. Q., et al., 2003, J Lipid Res. 44:2365-73).

The haplotype structure and frequencies were established among unrelated participants using Haplo.score (Schaid D. J., et al., 2002, Am J Hum Genet. 70:425-34). Selecting unrelated participants with complete data on all the analyzed variants (n=1,535), the four variants other than 56C>G variant were in almost complete linkage disequilibrium, with correlation coefficients ranging from 0.88 to 0.98, whereas the correlation coefficients between 56C>G variant and the other four variants ranged from 0.06 to 0.07 (Lai C. Q., et al., 2004, J Lip Res. 45:2096-105). There were a limited number of common haplotype variants (frequency>1%) (Table 3-A). When analyzing the whole sample, we could define the haplotype structures without ambiguous linkage phase in 2,047 of the 2,129 participants who had complete data on APOA5 genotypes. Three haplotype-genotype groups were defined (Table 3-B): haplotype-genotype APOA5*1/1, which includes homozygotes for the haplotype variant *1, the wild type, (n=1,578); haplotype-genotype APOA5*1/2 and 2/2, the carriers of the haplotype variant*2 (n=223); and haplotype-genotype APOA5*1/3 and 3/3, the carriers of the haplotype variant*3 (n-233). Because they were extremely infrequent (n=13), APOA5*2/3 heterozygous individuals were excluded from the association analyses as were those subjects with ambiguous or very rare haplotypes (n=82).

Other atherosclerosis risk factor variables: Data regarding the medical history and physical examination were derived from the 6th examination cycle. The following variables were included in the analyses: diabetes, smoking, hypertension, total cholesterol, LDL-cholesterol, HDL-cholesterol, triglycerides, height, weight, body mass index, waist circumference, obesity and abdominal obesity. C-Reactive Protein (CRP) levels were also determined as previously reported (Wang T. J., et al., 2002, Arterioscler Thromb Vasc Biol. 22:1662-7).

Statistical methods: Chi-square tests were used to compare proportions across groups, and analysis of variance to compare means of continuous variables across groups. Analysis of covariance was employed to determine the carotid IMT mean across APOA5 genotypes, adjusting for covariates. Logistic regression analysis was conducted to determine the association between genetic variants and ICA stenosis. Familial correlations were accounted for using generalized estimating equations with Proc Genmod in SAS (Version 8.0). For these analyses, a dominant genetic model was assumed, as the frequency of the rare alleles was low. In these models we adjusted for the following covariates: age, sex, smoking, diabetes, systolic blood pressure, hypertension treatment and body mass index, triglycerides, HDL-cholesterol and LDL-cholesterol. We ran three models, one adjusting for age and sex; a second adjusting for all covariates except the lipid measures; and a third with all covariates.

Additionally, haplotype analyses were performed in the subset of individuals with unambiguous linkage phase (2,047 of 2,129). According to the individual haplotype structures we defined three haplotype-genotypes using a dominant genetic model as previously explained (Table 3-B). In these analyses, we also employed generalized estimating equations and logistic regression with Proc Genmod in SAS as previously described.

We also tested for interactions between the genetic variants (individual variants and haplotypes) and hypertension, smoking, diabetes and obesity on carotid phenotypes. For all analyses, a two-tailed nominal p value less than 0.05 was considered statistically significant and we accounted for familial correlations.

The frequency of each of the different APOA5 rare alleles (analyzed in the unrelated subsample of the study) was 0.06 for 56C>G and IV53+476G>A variants, and 0.07 for −1131T>C, −3A>G and 1259T>C variants. These frequencies were consistent with Hardy-Weinberg equilibrium, except the 56C>G genetic variant). For 56C>G, there was a small but statistically significant difference between the observed genotype frequencies (89.1, 10.1 and 0.8, for the CC, CG and GG genotypes, respectively) and the expected frequencies (88.7, 11.0 and 0.3, respectively) in this large sample size. Given the very small magnitude of difference, we continued to include this variant in subsequent analyses.

In Table 4, we present the characteristics of the participants by each of the APOA5 genetic variants. With respect to lipids, there was a consistently higher triglyceride level associated with the presence of the rare allele in all of the analyzed APOA5 variants, with increases ranging from 22 to 27%. Compared with non-carriers, total cholesterol levels were higher in carriers of the rare allele in the −1131T>C, −3A>G and IVS3+476G>A variants and HDL cholesterol levels were lower in those with the rare allele of the 56C>G variant. Additionally, the prevalence of diabetes was lower among the carriers of the rare allele of the −1131T>C, −3A>G and IVS3+476G>A variants. The prevalence of obesity was lower among the carriers of the rare allele of the 1259T>C genetic variant.

There were no significant associations of the individual APOA5 genetic variants with carotid IMT or stenosis (Table 4). Similarly, there were no overall associations in age and sex-, and multivariable adjusted models. In analyses of association of haplotypes with carotid measures, the global test for differences across the three APOA5 haplotypes for CCA IMT was marginal (p=0.09). However, there were significant haplotype-specific differences: the APOA5*1/3-3/3 haplotype-genotype was associated with significantly higher CCA IMT compared with the APOA5*1/1 haplotype-genotype (p=0.040, Table 5). These significant differences persisted in multivariable-adjusted models, even after adjustment for triglycerides and other lipid levels. There were no associations between the different haplotype-genotype groups and ICA IMT or stenosis. These haplotype results were similar in analyses conducted separately in men and women.

We further analyzed whether other cardiovascular risk factors modulate the association of individual variants with carotid phenotypes. Significant interactions were consistently noted for BMI as well as a number of other obesity-related measures. BMI was directly associated with CCA IMT independently of the APOA5 genotype, but the association was significantly stronger in carriers of the rare alleles of the −1131T>C, −3A>G, IVS+476G>A and 1259T>C genetic variants. These interactions remained significant (p values ranging from 0.004 to 0.016) even after adjusting for triglycerides and other lipid levels. There was also a statistically significant interaction when we used waist circumference instead of BMI. In analyses to examine for interaction between the APOA5 genotypes and the dichotomous measure of obesity (BMI>30 kg/m2), each of the rare alleles was associated with higher CCA IMT in obese subjects (FIG. 8). Similar significant interactions were also noted with the dichotomous measure of abdominal obesity and were also observed in analyses conducted separately in men and women (data not shown).

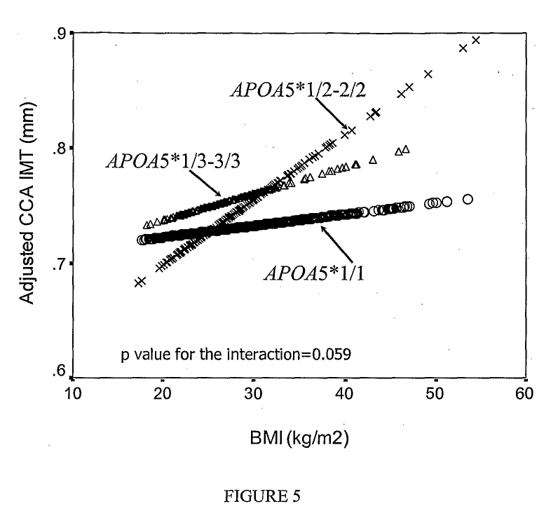

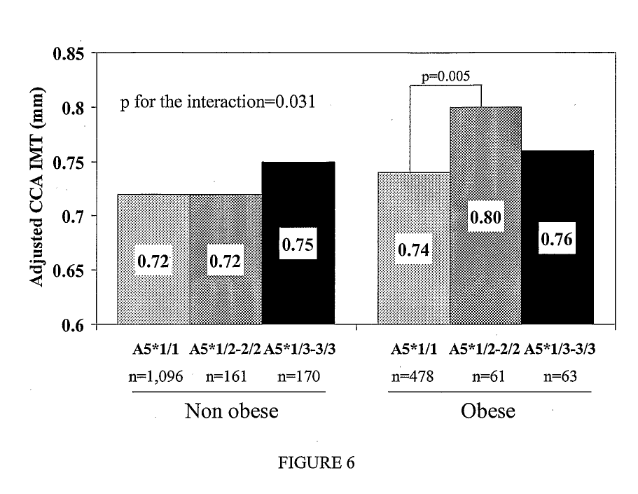

In analyses for interactions between the APOA5 haplotype variants and obesity related variables, the interaction between the APOA5 haplotype-genotypes and BMI was marginally significant (p=0.059) and the results were consistent with those noted for the individual genetic variants (FIG. 5). The association of BMI with CCA IMT was stronger in carriers of the APOA5*1/2-2/2 haplotype-genotype. The interaction between APOA5 haplotype-genotypes and obesity was statistically significant, even after adjusting for lipids (p=0.031): the APOA5*1/2-2/2 haplotype-genotype was associated with higher CCA IMT in obese people (FIG. 6). These interactions were also observed in analyses conducted separately in men and women. Aside from the obesity interactions, there were no other significant interactions of other risk factors with APOA5 variants.

Table 1 shows structure and frequency of the three common APOA5 haplotype variants (frequency>1%) (A), and frequency of the three common APOA5 haplotype-genotypes groups defined (B).

TABLE 3A
APOA5 Haplotype

| Variants | −1131T > C | −3A > G | | IVS + 476G > A | 1259T > C | Frequency |
|---|---|---|---|---|---|---|
| Variant *1 | T | A | C | G | T | 86.7 |
| Variant *2 | C | G | C | A | C | 6.0 |
| Variant *3 | T | A | G | G | T | 5.6 |
| Others | — | — | — | — | — | 1.7 |

TABLE 3B
APOA5 Haplotype-Genotype Groups

| | Haplotype-Genotype | N | Frequency |
|---|---|---|---|
| APOA5*1/1 | TACGT-TACGT | 1,578 | 74.11 |
| APOA5*1/2 or 2/2 | TACGT-CGCAC | 223 | 10.47 |
| | or | | |
| | CGCAC-CGCAC | | |
| APOA5*1/3 or 3/3 | TACGT-TAGGT | 233 | 10.95 |
| | or | | |
| | TAGGT-TAGGT | | |
| APOA5*2/3 | CGCAC-TAGGT | 13 | 0.61 |
| Other or | — | 82 | 3.85 |
| ambiguous | | | |

TABLE 4
Characteristics of the participants, common and internal carotid artery intimal medial
thickness and prevalence of carotid stenosis by individual APOA5 genotypes.

| | −1131T > C | | | | −3A > G | | | | 56C > G | | | |
|---|---|---|---|---|---|---|---|---|---|---|---|---|
| | | | C | | | | G | | | | G | |
| | TT | | carriers | | AA | | carriers | | CC | | carriers | |
| | N = 1,971 | | N = 296 | | N = 2,026 | | N = 310 | | N = 2,084 | | N = 277 | |
| Age (years)^{a} | 58 | (10) | 58 | (10) | 58 | (10) | 59 | (10) | 59 | (10) | 57 | (10) |
| Women (%) | 53.0 | | 49.0 | | 53.0 | | 48.7 | | 52.7 | | 50.5 | |
| Smoking (%) | 15.0 | | 13.2 | | 14.6 | | 14.2 | | 15.1 | | 11.9 | |
| Diabetes (%) | 12.8 | | 8.5^{b} | | 12.8 | | 7.7^{b} | | 12.0 | | 12.6 | |
| Hypertension (%) | 42.0 | | 37.5 | | 41.2 | | 38.4 | | 40.8 | | 40.4 | |
| BMI (kg/m²)^{a} | 28.1 | (5.3) | 28.0 | (5.7) | 28.1 | (5.2) | 28.1 | (5.7) | 28.1 | (5.3) | 27.9 | (4.6) |
| Obesity (%) | 29.9 | | 25.4 | | 29.9 | | 26.2 | | 29.7 | | 26.4 | |
| Waist (mm)^{a} | 98 | (14) | 98 | (14) | 98 | (14) | 98 | (14) | 98 | (14) | 98 | (13) |
| Abd. Obesity | 49.5 | | 45.0 | | 49.5 | | 45.9 | | 48.7 | | 52.2 | |
| (%) | | | | | | | | | | | | |
| TC (mg/dL)^{a} | 205 | (37) | 212 | (41)^{c} | 205 | (36) | 212 | (40)^{c} | 206 | (37) | 206 | (38) |
| LDL-C | 128 | (32) | 129 | (35) | 127 | (32) | 130 | (35) | 127 | (33) | 125 | (33) |
| (mg/dL)^{a} | | | | | | | | | | | | |
| HDL-C | 51 | (16) | 50 | (16) | 51 | (16) | 50 | (16) | 51 | (16) | 48 | (15)^{c} |
| (mg/dL)^{a} | | | | | | | | | | | | |
| TG (mg/dL)^{a} | 137 | (91) | 172 | (135)^{c} | 136 | (90) | 172 | (131)^{c} | 137 | (93) | 167 | (132)^{c} |
| CCA IMT | 0.73 | (0.18) | 0.74 | (0.16) | 0.73 | (0.18) | 0.74 | (0.17) | 0.73 | (0.17) | 0.73 | (0.26) |
| (mm)^{a} | | | | | | | | | | | | |
| ICA IMT | 0.79 | (0.52) | 0.79 | (0.52) | 0.78 | (0.51) | 0.80 | (0.52) | 0.79 | (0.51) | 0.75 | (0.50) |
| (mm)^{a} | | | | | | | | | | | | |

| | | | | | | |
|---|---|---|---|---|---|---|
| Stenosis > 25% | 18.4 | 19.6 | 18.1 | 21.3 | 18.8 | 16.3 |
| (%) | | | | | | |

| | |
|---|---|
| IVS + 476G > A | 1259T > C |

| | | | |
|---|---|---|---|
| | A | | C |
| GG | carriers | TT | carriers |
| n = 2,021 | N = 273 | N = 2,009 | N = 314 |

| Age (years)^{a} | 58 | (10) | 58 | (10) | 58 | (10) | 58 | (10) |
|---|---|---|---|---|---|---|---|---|
| Women (%) | 52.9 | | 49.1 | | 53.4 | | 49.4 | |
| Smoking (%) | 15.0 | | 13.9 | | 14.9 | | 14.7 | |
| Diabetes (%) | 12.7 | | 7.7^{b} | | 12.4 | | 9.6 | |
| Hypertension (%) | 41.3 | | 38.1 | | 41.5 | | 38.5 | |
| BMI (kg/m²)^{a} | 28.1 | (5.2) | 28.0 | (5.7) | 28.1 | (5.3) | 27.8 | (5.6) |
| Obesity (%) | 30.0 | | 26.1 | | 30.1 | | 24.6^{b} | |
| Waist (mm)^{a} | 98 | (14) | 98 | (14) | 98 | (14) | 98 | (14) |
| Abd. Obesity | 49.7 | | 45.9 | | 49.8 | | 43.7 | |
| (%) | | | | | | | | |
| TC (mg/dL)^{a} | 205 | (36) | 211 | (40)^{b} | 205 | (36) | 209 | (40) |
| LDL-C | 127 | (32) | 130 | (36) | 127 | (32) | 128 | (35) |
| (mg/dL)^{a} | | | | | | | | |
| HDL-C | 51 | (16) | 50 | (16) | 51 | (16) | 50 | (16) |
| (mg/dL)^{a} | | | | | | | | |
| TG (mg/dL)^{a} | 136 | (91) | 172 | (132)^{c} | 136 | (92) | 169 | (128)^{c} |
| CCA IMT | 0.73 | (0.18) | 0.74 | (0.17) | 0.73 | (0.18) | 0.74 | (0.16) |
| (mm)^{a} | | | | | | | | |
| ICA IMT | 0.78 | (0.51) | 0.78 | (0.51) | 0.78 | (0.51) | 0.79 | (0.51) |
| (mm)^{a} | | | | | | | | |

| | | | | |
|---|---|---|---|---|
| Stenosis > 25% | 18.0 | 19.4 | 18.2 | 20.1 |
| (%) | | | | |

Abbreviations:
BMI = Body mass index;
Abd. obesity = Abdominal obesity;
TC = Cholesterol;
LDL = Low density lipoproteins;
HDL = High density lipoproteins;
TG = Triglycerides,
CCA IMT = Common carotid artery intimal medial thickness;
ICA IMT = Internal carotid artery intimal medial thickness.;
^{a}Continuous variables are presented as mean (standard deviation);
^{b}p < 0.05;
^{c}p < 0.01(for triglycerides p value is based on log triglycerides).

TABLE 5
Common and internal carotid intimal medial thickness [mean (standard
error)], and odds ratios (95% confidence interval) for prevalence
of carotid stenosis across APOA5 haplotype-genotype groups.

| | | | |
|---|---|---|---|
| | APOA5*1/2-2/2 | APOA5*1/3-3/3 | |
| | (T-A-C-G-T/ | (T-A-C-G-T/ | |
| | C-G-C-A-C | T-A-G-G-T | |
| APOA5*1/1 | or | or | |
| (T-A-C-G-T/ | C-G-C-A-C/ | T-A-G-G-T/ | |
| T-A-C-G-T) | C-G-C-A-C) | T-A-G-G-T) | |
| n = 1,578 | n = 223 | n = 233 | P |

Common Carotid Artery Intimal Medial Thickness (mm)

| | | | | |
|---|---|---|---|---|
| Unadjusted | 0.73 (0.01) | 0.74 (0.01) | 0.74 (0.01) | 0.610 |
| Model 1 ^{a} | 0.73 (0.00) | 0.74 (0.01) | 0.75 (0.01) | 0.242 |
| Model 2 ^{a} | 0.73 (0.00) | 0.74 (0.01) | 0.75 (0.01) ^{b} | 0.092 |
| Model 3 ^{a} | 0.73 (0.01) | 0.74 (0.01) | 0.75 (0.01) ^{c} | 0.091 |

Internal Carotid Artery Intimal Medial Thickness (mm)

| | | | | |
|---|---|---|---|---|
| Unadjusted | 0.78 (0.01) | 0.80 (0.03) | 0.76 (0.03) | 0.643 |
| Model 1 | 0.78 (0.00) | 0.80 (0.03) | 0.77 (0.03) | 0.789 |
| Model 2 | 0.77 (0.00) | 0.81 (0.03) | 0.78 (0.03) | 0.496 |
| Model 3 | 0.77 (0.01) | 0.81 (0.03) | 0.76 (0.03) | 0.510 |

Internal Carotid Artery Stenosis >25%

| | | | |
|---|---|---|---|
| Unadjusted | 1 | 1.00 (0.69-1.44) | 0.81 (0.55-1.18) |
| Model 1 | 1 | 1.04 (0.72-1.50) | 0.87 (0.58-1.31) |
| Model 2 | 1 | 1.19 (0.83-1.71) | 0.92 (0.60-1.41) |
| Model 3 | 1 | 1.18 (0.80-1.74) | 0.94 (0.61-1.43) |

^{a} Model 1: Age and sex adjusted; Model 2: Age, sex, smoking, diabetes, systolic blood, hypertension treatment, body mass index; Model 3: Model 2 plus further adjustment for triglycerides, HDL-cholesterol and LDL-cholesterol.
^{b} p = 0.041 compared to Haplotype-genotype APO45*1/1.
^{c} p = 0.040 compared to Haplotype-genotype APOA5*1/1.

The references cited herein and throughout the specification are herein incorporated by reference in their entirety.
1. Zilversmit D B. Atherogenesis: a postprandial phenomenon. Circulation 1979; 60:473-85.
2. Karpe F. Postprandial lipoprotein metabolism and atherosclerosis. J Intern Med 1999; 246:341-355.
3. Ordovas J M. Genetics, postprandial lipemia and obesity. Nutr Metab Cardiovasc Dis. 2001; 11:118-33.
4. McNamara J R, Shah P K, Nakajima K, Cupples L A, Wilson P W, Ordovas J M, Schaefer E J. Remnant-like particle (RLP) cholesterol is an independent cardiovascular disease risk factor in women: results from the Framingham Heart Study. Atherosclerosis. 2001; 154:229-236
5. McNamara J R, Shah P K, Nakajima K, Cupples L A, Wilson P W, Ordovas J M, Schaefer E J. Remnant lipoprotein cholesterol and triglyceride reference ranges from the Framingham Heart Study. Clin Chem. 1998; 44:1224-32.
6. Masuoka H, Kamei S, Wagayama H, Ozaki M, Kawasaki A, Tanaka T, Kitamura M, Katoh S, Shintani U, Misaki M, Sugawa M, Ito M, Nakano T. Association of remnant-like particle cholesterol with coronary artery disease in patients with normal total cholesterol levels. Am Heart J. 2000; 139:305-10.
7. Karpe F, Taskinen M R, Nieminen M S, Frick M H, Kesaniemi Y A, Pasternack A, Hamsten A, Syvanne M. Remnant-like lipoprotein particle cholesterol concentration and progression of coronary and vein-graft atherosclerosis in response to gemfibrozil treatment. Atherosclerosis. 2001; 157:181-7.
8. Kawakami A, Yoshida M. Remnant lipoproteins and atherogenesis. J Atheroscler Thromb. 2005; 12:73-6.
9. Pennacchio L A, Olivier M, Hubacek J A, Cohen J C, Cox D R, Fruchart J C, Krauss R M, Rubin E M. An apolipoprotein influencing triglycerides in humans and mice revealed by comparative sequencing. Science. 2001; 294:169-173.
10. van der Vliet H N, Sammels M G, Leegwater A C, Levels J H, Reitsma P H, Boers W, Chamuleau R A. Apolipoprotein A-V: a novel apolipoprotein associated with an early phase of liver regeneration. J Biol Chem. 2001; 276:44512-20.
11. Weinberg R B, Cook V R, Beckstead J A, Martin D D, Gallagher J W, Shelness G S, Ryan R O. Structure and interfacial properties of human apolipoprotein A-V. J Biol Chem. 2003; 278:34438-44.
12. Schaap F G, Rensen P C, Voshol P J, Vrins C, van der Vliet H N, Chamuleau R A, Havekes L M, Groen A K, van Dijk K W. ApoAV reduces plasma triglycerides by inhibiting very low density lipoprotein-triglyceride (VLDL-TG) production and stimulating lipoprotein lipase-mediated VLDL-TG hydrolysis. J Biol Chem. 2004; 279:27941-7.
13. Fruchart-Najib J, Bauge E, Niculescu L S, Pham T, Thomas B, Rommens C, Majd Z, Brewer B, Pennacchio L A, Fruchart J C. Mechanism of triglyceride lowering in mice expressing human apolipoprotein A5. Biochem Biophys Res Commun. 2004; 319:397-404
14. Merkel M, Loeffler B, Kluger M, Fabig N, Geppert G, Pennacchio L A, Laatsch A, Heeren J. Apolipoprotein AV accelerates plasma hydrolysis of triglyceride-rich lipoproteins by interaction with proteoglycan-bound lipoprotein lipase. J Biol Chem. 2005; 280:21553-60.
15. Pennacchio L A, Olivier M, Hubacek J A, Krauss R M, Rubin E M, Cohen J C. Two independent apolipoprotein A5 haplotypes influence human plasma triglyceride levels. Hum Mol Genet. 2002; 11:3031-3038.
16. Talmud P J, Hawe E, Martin S, Olivier M, Miller G J, Rubin E M, Pennacchio L A, Humphries S E. Relative contribution of variation within the APOC3/A4/A5 gene cluster in determining plasma triglycerides. Hum Mol Genet. 2002; 11:3039-3046.
17. Nabika T, Nasreen S, Kobayashi S, Masuda J. The genetic effect of the apoprotein AV gene on the serum triglyceride level in Japanese. Atherosclerosis. 2002; 165:201-204.
18. Lai C Q, Tai E S, Tan C E, Cutter J, Chew S K, Zhu Y P, Adiconis X, Ordovas J M. The apolipoprotein A5 locus is a strong determinant of plasma triglyceride concentrations across ethnic groups in Singapore. J Lipid Res. 2003; 44: 2365-2373.
19. Lai C Q, Demissie S, Cupples L A, Zhu Y, Adiconis X, Parnell L D, Corella D, Ordovas J M. Influence of the APOA5 locus on plasma triglyceride, lipoprotein subclasses, and CVD risk in the Framingham Heart Study. J Lipid Res 2004; 45:2096-105.
20. Nakamura M T, Cheon Y, Li Y, Nara T Y. Mechanisms of regulation of gene expression by fatty acids. Lipids. 2004; 39:1077-1083
21. Ascherio A, Rimm E B, Stampfer M J, Giovannucci E L, Willett W C. Dietary intake of marine n-3 fatty acids, fish intake, and the risk of coronary disease among men. N Engl J Med. 1995; 332:977-982.
22. Morris M C, Manson J E, Rosner B, Buring J E, Willett W C, Hennekens C H. Fish consumption and cardiovascular disease in the Physicians' Health Study: a prospective study. Am J Epidemiol. 1995; 142:166-175.
23. Leaf A, Kang J X, Xiao Y F, Billman G E. Clinical Prevention of Sudden Cardiac Death by n-3 Polyunsaturated Fatty Acids and Mechanism of Prevention of Arrhythmias by n-3 Fish Oils. Circulation. 2003; 107:2646-2652.
24. Siscovick D S, Lemaitre R N, Mozaffarian D. The Fish Story-A Diet-Heart Hypothesis With Clinical Implications: n-3 Polyunsaturated Fatty Acids, Myocardial Vulnerability, and Sudden Death. Circulation 2003; 107:2632
25. de Lorgeril M, Salen P, Martin J L, Monjaud I, Delaye J, Mamelle N. Mediterranean diet, traditional risk factors, and the rate of cardiovascular complications after myocardial infarction: final report of the Lyon Diet Heart Study. Circulation. 1999; 99:779-85.
26. Feinleib M, Kannel W B, Garrison R J, McNamara P M, Castelli W P. The Framingham Offspring Study. Design and preliminary data. Prev Med. 1975; 4:518-25.
27. Cupples L A, Gagnon D R, Kannel W B. Long- and short-term risk of sudden coronary death. Circulation. 1992; 85:i11-i18.
28. Freedman D S, Otvos J D, Jeyarajah E J, Barboriak J J, Anderson A J, Walker J A. Relation of lipoprotein subclasses as measured by proton nuclear magnetic resonance spectroscopy to coronary artery disease. Arterioscler Thromb Vasc Biol. 1998; 18:1046-1053.
29. Rimm E B, Giovannucci E L, Stampfer M J, Colditz G A, Litin L B, Willett W C. Reproducibility and validity of an expanded self-administered semiquantitative food frequency questionnaire among male health professionals. Am. J. Epidemiol. 1992; 135:1114-1126.
30. Hunter D J, Rimm E B, Sacks F M, Stampfer M J, Colditz G A, Litin L B, Willett W C. Comparison of measures of fatty acid intake by subcutaneous fat aspirate, food frequency questionnaire, and diet records in a free-living population of US men. Am J Epidemiol. 1992; 135:418-427.
31. Tai E S, Corella D, Demissie S, Cupples L A, Coltell O, Schaefer E J, Tucker K L, Ordovas J M. Polyunsaturated fatty acids interact with the PPARA-L162V polymorphism to affect plasma triglyceride and apolipoprotein C-III concentrations in the Framingham Heart Study. J Nutr. 2005; 135:397-403.
32. Williams K J, Tabas I. The response-to-retention hypothesis of atherogenesis reinforced. Curr Opin Lipidol 1998; 9:471-4.
33. Simopoulos A P. Essential fatty acids in health and chronic disease. Am J Clin Nutr 1999; 70:560 S-9S.
34. Dwyer J H, Allayee H, Dwyer K M, Fan J, Wu H, Mar R, Lusis A J, Mehrabian M. Arachidonate 5-lipoxygenase promoter genotype, dietary arachidonic acid, and atherosclerosis. N Engl J Med. 2004; 350:29-37.
35. Marcoux C, Tremblay M, Fredenrich A, Jacques H, Krimbou L, Nakajima K, Davignon J, Colin J S. Plasma remnant-like particle lipid apolipoprotein levels in normolipidemic and hyperlipidemic subjects. Atherosclerosis 1998; 139: 161-71.
36. Talmud P J, Palmen J, Putt W, Lins L, Humphries S E. Determination of the functionality of common APOA5 polymorphisms. J Biol Chem. 2005; 10.1074/jbc.M502144200.
37. Jang Y, Kim J Y, Kim O Y, Lee J E, Cho H, Ordovas J M, Lee J H. The −1131T->C polymorphism in the apolipoprotein A5 gene is associated with postprandial hypertriacylglycerolemia; elevated small, dense LDL concentrations; and oxidative stress in nonobese Korean men. Am J Clin Nutr. 2004; 80: 832-840.
38. Martin S, Nicaud V, Humphries S E, Talmud P J. On behalf of the EARS group. Contribution of APOA5 gene variants to plasma triglyceride determination and to the response to both fat and glucose tolerance challenges. Biochim Biophys Acta. 2003; 1637:217-225.
39. Masana L, Ribalta J, Salazar J, Fernandez-Ballart J, Joven J, Cabezas M C. The apolipoprotein AV gene and diurnal triglyceridaemia in normolipidaemic subjects. Clin Chem Lab Med. 2003; 41:517-21.

We claim:

1. A method for screening for an individual who is at risk of an altered lipid profile when consuming n-6 polyunsaturated fatty acids, the method comprising determining APOA5 locus genotype in a biological sample taken from the individual, wherein presence of one or two of any of the alleles selected from the group consisting of APOA5−1131C, APOA5−3G, APOA5 IVS3+476A, and APOA5 1259C alleles or alleles that are found to be in tight linkage disequilibrium with APOA5−1131C, APOA5−3G, APOA5 IVS3+476A, and APOA5 1259C alleles, or any combination thereof is indicative of an individual being at risk of the altered lipid profile.

2. The method of claim 1, wherein the altered lipid profile is an atherogenic lipid profile.

3. The method of claim 2, wherein the atherogenic lipid profile includes increased plasma remnant-like particle (RLP) concentration, and increased plasma very low density lipoprotein (VLDL) size and decreased plasma low density lipoprotein (LDL) size.

4. A kit for providing dietary advice to an individual comprising:
a) a system for genotyping APOA5 locus for at least one polymorphic marker from a biological sample; and
b) instructions that if one or two of alleles selected from the group consisting of APOA5−1131C, APOA5−3G, APOA5 IVS3+476A, and APOA5 1259C alleles is detected, the individual is advised to limit or avoid consumption of foods containing n-6 polyunsaturated fatty acids to avoid developing an atherogenic lipid profile, and that if no alleles selected from the group consisting of APOA5−1131C, APOA5−3G, APOA5 IVS3+476A, and APOA5 1259C or that the presence of a homozygous allele selected from the group consisting of APOA5−1131T, APOA5−3A, APOA5 IVS3+476G, and APOA5 1259T alleles is detected, the individual can consume n-6 polyunsaturated fatty acids without being at increased risk of developing an atherogenic lipid profile.

5. A kit for providing dietary advice to an individual comprising:
a) a system for genotyping APOA5 locus for at least one polymorphic marker from a biological sample; and
b) instructions that if one or two of alleles selected from the group consisting of APOA5−1131C, APOA5−3G, APOA5 IVS3+476A, and APOA5 1259C alleles is detected, the individual is advised to limit or avoid consumption of foods containing n-6 polyunsaturated fatty acids to avoid developing an atherogenic lipid profile.

6. A kit for providing dietary advice to an individual comprising:
a) a system for genotyping APOA5 locus for at least one polymorphic marker from a biological sample; and
b) instructions that if no alleles selected from the group consisting of APOA5−1131C, APOA5−3G, APOA5 IVS3+476A, and APOA5 1259C or that the presence of a homozygous allele selected from the group consisting of APOA5−1131T, APOA5−3A, APOA5 IVS3+476G, and APOA5 1259T alleles is detected, the individual can consume n-6 polyunsaturated fatty acids without being at increased risk of developing an atherogenic lipid profile.

7. The kit of claim 4, wherein the system for genotyping APOA5 locus comprises attached nucleic acid probes on a solid surface.

8. The kit of claim 7, wherein the solid surface is selected from chips and beads.

9. A kit for screening for an individual at risk of atherogenic plasma lipid profile when exposed to polyunsaturated fatty acids, wherein the kit comprises a plurality of isolated oligonucleotides, the oligonucleotides corresponding to no more than about 100 polymorphisms, wherein at least one of the polymorphisms is selected from the group consisting of APOA5−1131C, APOA5−3G, APOA5 IVS3+476A, and APOA5 1259C, and a guideline that indicates that if any of the polymorphic alleles consisting of APOA5−1131C, APOA5−3G, APOA5 IVS3+476A, and APOA5 1259C is detected in a biological sample from an individual, the individual should be advised to avoid n-6 polyunsaturated fatty acids.

10. The kit of claim 9, wherein the plurality of isolated oligonucleotides are attached on a solid surface.

11. The kit of claim 10, wherein the sold surface is selected from nucleic acid chips and beads.

12. A method for creating diet advice comprising:
a) providing a service to screen for at least one of the polymorphisms selected from the group consisting of APOA5−1131C, APOA5−3G, APOA5 IVS3+476A, and APOA5 1259C in a biological sample from an individual;
b) providing a service to create a diet advice with restricted amount of n-6 polyunsaturated fatty acids for the individual whose sample contains at least one allele of APOA5−1131C, APOA5−3G, APOA5 IVS3+476A, or APOA5 1259C; and
c) delivering the diet advice to the individual.

13. A method for creating diet advice comprising:
a) providing a service to create diet advice with a restricted amount of n-6 polyunsaturated fatty acids for an individual whose sample contains at least one allele selected from the group consisting of APOA5−113 IC₅ APOA5−3G, APOA5 IVS3+476A, and APOA5 1259C; and
b) delivering the diet advice to the individual.
